(19) United States
(12) Patent Application Publication
(10) Pub. No.: US 2012/0165340 A1
(43) Pub. Date: Jun. 28, 2012

(54) PTEN PHOSPHORYLATION-DRIVEN RESISTANCE TO CANCER TREATMENT AND ALTERED PATIENT PROGNOSIS

(75) Inventors: Frank Furnari, La Jolla, CA (US); Webster Cavenee, La Jolla, CA (US); Timothy Fenton, La Jolla, CA (US)

(73) Assignee: Ludwing Institute for Cancer Research Ltd., New York, NY (US)

(21) Appl. No.: 13/138,645

(22) PCT Filed: Feb. 11, 2010
(86) PCT No.: PCT/US2010/000387

§ 371 Date: Feb. 17, 2012

Related U.S. Application Data
(60) Provisional application No. 61/207,364, filed on Feb. 11, 2009.

(51) Int. Cl. A61K 31/496; G01N 27/62; A61P 35/00; C12Q 1/02; A61K 31/506; C07K 16/18

(52) U.S. Cl. 514/252.19; 514/275; 530/389.1; 530/388.2; 435/29; 435/7.1

(57) ABSTRACT

Indicators that can guide clinical decisions in cancer, particularly posttranslational modification of proteins which result in altered prognosis and differential sensitivity to targeted cancer therapy, are provided. In particular, monitoring of phosphorylation of PTEN may be utilized to predict or assess drug response, drug sensitivity, and clinical outcome. Modulation of PTEN phosphorylation may be utilized to alter sensitivity and outcome in cancer patients. Posttranslational modification of PTEN, particularly phosphorylation, is correlated with resistance to targeted cancer therapy, including EGFR inhibitors, and with reduced survival prognosis. Methods and assays for determining phosphorylation of PTEN, particularly Y240 phosphorylation, are provided. Methods for sensitizing tumors to inhibition and targeted therapy by modulating PTEN phosphorylation are provided.

### FIELD OF THE INVENTION

The present invention relates to indicators that can guide clinical decisions in treatment and diagnosis of cancer, particularly posttranslational modification of proteins which result in altered survival prognosis and differential sensitivity to targeted cancer therapy. In particular, monitoring of phosphorylation of PTEN may be utilized to predict or assess drug response, drug sensitivity, and clinical outcome, and modulation of PTEN phosphorylation may be utilized to alter drug sensitivity and outcome in cancer patients. The present invention relates to post-translational modification of PTEN, particularly phosphorylation, and its correlation with resistance to targeted cancer therapy, including EGFR inhibitors, and with reduced survival prognosis. Methods and assays for determining phosphorylation of PTEN, particularly Y240 phosphorylation, are provided. Methods for sensitizing tumors to inhibition and targeted therapy by modulating PTEN phosphorylation are provided.

### BACKGROUND OF THE INVENTION

Oncologists have a number of treatment options available to them, including different combinations of chemotherapeutic drugs that are characterized as “standard of care,” and a number of drugs that do not carry a label claim for the treatment of a particular cancer, but for which there is evidence of efficacy in that cancer. The highest likelihood of good treatment outcome requires that patients at highest risk of metastatic disease, with poorest prognosis, or with specific cancer marker characteristics be identified and assigned to optimal available cancer treatment. In particular, it is important to determine the likelihood of patient response to “standard of care” therapeutic drugs, such as cyclophosphamide, methotrexate, 5-fluorouracil, anthracyclines, taxanes, and to more target-directed therapeutics, such as kinase inhibitors (such as gefitinib, erlotinib, dasatinib) and antibody therapy (such as ErbB2 (Herceptin) and EGFR (Cetuximab)), because these have varied efficacy and a spectrum of side effects and rates of development of resistance. The identification of patients who are most or least likely to respond to available drugs thus could increase the net benefit these drugs have to offer, and decrease net morbidity and toxicity, via more intelligent patient and drug selection, including the continued monitoring of the status of these patients and appropriate adjustment of their therapy. Acquired resistance to chemotherapy or targeted cancer therapy, mediated by secondary resistance or compensatory mutations is an ongoing challenge.

Targeted cancer therapy is designed to disrupt the function of specific molecules needed for carcinogenesis and tumor growth and thus either specifically kills or prevents the growth of cancer cells (Ji H et al (2006) Cell Cycle 5(18):2072-2076 Epub 2006 Sep. 15). In contrast to conventional cytotoxic chemotherapy, such targeted cancer therapies may be more effective and less harmful to normal cells. One effort in the targeted cancer therapy field has been the development of agents that target the epidermal growth factor receptor (EGFR). EGFR is a member of the ErbB family of closely related receptors including EGFR (ErbB-1), Her2/neu (ErbB-2), Her3 (ErbB-3) and Her4 (ErbB-4). The ErbB family of receptors are tyrosine kinase cell surface receptors and their extracellular domains are appropriate targets for modulators and antibody therapies. Activation of EGFR leads to receptor tyrosine kinase activation and a series of downstream signaling events that mediate cellular proliferation, motility, adhesion, invasion, and resistance to chemotherapy as well as inhibition of apoptosis, processes that are crucial to the continual proliferation and survival of cancer cells. EGFR is over-expressed or mutated in many types of epithelial tumors. Moreover, expression of the EGFR is associated with poor prognosis in a number of tumor types including stomach, colon, urinary bladder, breast, prostate, endometrium, kidney and brain (e.g., glioma). ErbB2 is found in approximately 25% of breast cancer and ErbB3 is found in 80% of GI tract tumors. More broadly, tyrosine kinases are implicated in many cancers based on their pivotal roles in many cell-signalling processes.

To date, two major types of anti-EGFR agents have entered the clinical setting: anti-EGFR antibodies and small molecule EGFR tyrosine kinase inhibitors (TKIs) (5, 6). Anti-EGFR antibodies such as cetuximab were designed to bind to the extra-cellular domain of the EGFR and block activation of EGFR downstream signaling (7). Cetuximab (also known as antibody 225, U.S. Pat. No. 4,943,533) was raised against A431 cells, which express high levels of wild type EGFR. In contrast, small molecule TKIs such as gefitinib (compound ZD1839, Iressa) or erlotinib (compound OS1-774, Tarceva) compete with ATP for binding to the intracellular catalytic domain of the EGFR tyrosine kinase and, thus, prevent EGFR autophosphorylation and downstream signaling (4).

Both anti-EGFR antibodies and EGFR tyrosine kinase inhibitors have shown some clinical efficacy in a subset of patients with a variety of different types of cancers. Treatment with gefitinib or erlotinib in patients with lung cancer having EGFR kinase domain mutations often generate dramatic clinical responses (5, 8). However, the effectiveness of gefitinib or erlotinib in lung adenocarcinoma with wild type EGFR or in other histological subtype, such as squamous cell carcinoma is limited (9, 10). Furthermore, it has been shown in pre-clinical and clinical trials that gefitinib or erlotinib are largely ineffective in inhibiting the function of the EGFRvIII mutant (11), a distinct activating EGFR mutation in which there is an in-frame deletion of exon 2 to 7 (also denoted EGFR de2-7). EGFRvIII is commonly found in glioblastomas and recently found to be present in a subset of human lung squamous cell carcinomas (12) and a large fraction of head and neck cancers (13).

Phosphatase and tensin homolog (PTEN) is a tumor suppressor gene mutated in many human cancers. The major substrate of its lipid phosphatase activity is PIP3. By dephosphorylating PIP3, PTEN negatively regulates the PI3K pathway and AKT activation and thus suppresses tumorigenesis. PI3K is a downstream effector of the EGFR cascade. PTEN is the second most frequently mutated tumor suppressor gene in human sporadic cancer and reduced PTEN expression occurs in approximately half of all tumors (Suzuki, A. et al (2008) Cancer Sci 99(2): 209-213). PTEN has been implicated to play a role in glioblastoma, melanoma, breast, prostate, renal, endometrial, colon, lung, and thyroid cancer (Tamguney, T. and Stokoe, D. (2007) J. Cell Sci 12: 4071-4079; Koul, D. (2008) Cancer Biology & Therapy 7(9): 1321-1325; Soria, J C, Siena, S. et al (2009) J. Natl Cancer Inst 101: 1304-1324). Reduced PTEN expression (including null) or PTEN activation have been reported to correlate with altered drug sensitivity, including to EGFR monoclonal antibodies (eg Panitumumab, Cetuximab), ErbB2 antibody (Trastuzumab) to PI3 Kinase/AKT inhibitors, and other receptor tyrosine kinase inhibitors (Siena, S. et al (2009) J. Natl Canc Inst 101:1308-1324; Nagata, y et al (2007) Cancer Cell 6:117-127; Ming, M and He Y-Y (2009) J Invest Dermatol 129(9): 2109-2112; de Graffenreid L. A. et al (2004) Annals of Oncology 15:1510-1516; Abounader, R. (2009) Expect Rev Anticancer Ther 9(2): 235-245); Loupkis, F et al (2008) J Clin Oncol 26(May 20 Suppl) abst 4003).

The PTEN tumor suppressor gene is lost or mutated in approximately 40% of glioblastomas (GBMs), leading to aberrant activation of highly oncogenic PI3K/Akt signaling and other downstream pathways^{1, 6}. In addition to this regulatory function, it is becoming increasingly clear that, even in cells harboring the wild type PTEN gene, its protein function can be affected by a number of posttranslational modifications, including phosphorylation, acetylation and ubiquitination⁷. This complicates the significance of detecting wild type PTEN in tumor cells, as it is not necessarily an indication that PTEN function has been retained. This lends increasing importance to being able to detect modified PTEN in human tumors and to discover the molecules that, as PTEN-modifiers, may represent useful therapeutic targets to restore PTEN tumor suppressor function. Phosphorylation of PTEN by src-family kinases has been proposed to inhibit its function in a number of ways, including loss of membrane interaction and decreased protein stability^{8, 9}, but it is not clear how this is accomplished and whether these modifications have any import for the behavior of human tumors.

The non-receptor protein tyrosine, Src, is a 60-kDa protein that is a member of a nine-gene family, including Src, Yes, Fyn, Lyn, Lck, Hck, Fgr, Blk, and Yrk. Src plays a critical role in the regulation of many cellular processes, such as proliferation, differentiation, migration, adhesion, invasion, angiogenesis, and immune function (Yeatman T J. (2004) Nat Rev Cancer 4(6):470-80; Frame M C. (2004) J Cell Sci 117:989-98). The Src family kinase contains a poorly conserved domain and three conserved Src homology domains: SH2, SH3, and SH1 or protein tyrosine kinase domain. Critical to the regulation of Src is a COOH-terminal tyrosine (Y530) that, when phosphorylated by C-terminal Src kinase (Csk), leads to a more inactive Src conformation. Src interacts with many proteins, depending on the input signal. It further assumes its active conformation through dephosphorylation of Y530 and autophosphorylation of Y418. Src also associates with structural and signaling proteins, and the resulting complexes are critical to Src's role in diverse cellular processes. Src has been reported to be overexpressed or aberrantly activated in a number of cancers, such as colon, breast, melanomas, ovarian cancer, gastric cancer, head and neck cancers, pancreatic cancer, lung cancer, brain cancers, and blood cancers (Dehm S M and Bonham K (2004) Biochem Cell Biol 2004; 82:263-74). There are several known small molecule inhibitors of src and some have entered clinical trials, for example dasatinib (BMS354825), AZD-0530, SKI-606, PP1 (4-Amino-5-(4-methylphenyl)-7-(t-butyl)pyrazolo[3,4-d]-pyrimidine), PP2 (4-chlorophenyl)-7-(t-butyl)pyrazolo[3,4-d]-pyrimidine), PD166326.

Despite the similar domain structure of the Src family members, recent evidence suggests that individual Src family members may promote specific cellular functions, although the mechanism accounting for this is not yet entirely clear. Some evidence suggests that the SH4-unique-SH3-SH2 domains dictate specificity in signaling and other studies point to preferential association with specific cell surface receptors (Summy J M et al (2003) J Cell Sci 116:2585-2598; Kralisz U et al (1998) 45:735-743; Briddon S J et al (1999) Biochem J 338:203-209). Fyn has been shown to promote oligodendrocyte differentiation in brain and hemidesmosome disassembly in A431 epidermoid squamous carcinoma cells (Osterhout D J et al (1999) J Cell Biol 145:1209-1218; Wolf R M et al (2001) J Neurobiol 49:62-78; Mariotti A et al (2001) J Cell Biol 155:447-458). Elevated levels of c-Src have been reported in breast and colon cancer (Bolen J B et al (1987) PNAS USA 87:2251-2255; Egan C et al (1989) J Clin Invest 86:2025-2033; Irby R B et al (1999) Nat Genet. 21:187-190; Han N M et al (1996) Cancer Res 2:1397-1404; Verbeek B S et al (1996) J Pathol 180:383-388).

Lyn kinase activity is reported to be significantly elevated in glioblastoma tumor samples and accounted for over 90% of the pan-Src kinase activity (Stettner M R et al (2003) Cancer Res 65(13):5535-5543). Activation of Lyn is associated with CML and ALL patient relapse and resistance to Imatinib (Gleevec, STI571) (Wu J et al (2008) J Natl Cancer Inst 100(13):926-939; Donato N J et al (2008) Blood 101:690-698; Dai Y et al (2004) J Biol Chem 279:34227-34239). INNO-406 (formerly NS-187) was recently developed as a potent and selective dual BCR-ABL/Lyn inhibitor and inhibits Lyn without affecting the phosphorylation of Src-family kinases Src, Blk or Yes (Kinura S et al (2005) Blood 106:3928-3954). INNO-406 shows concentrations in the CNS of about 10% of those in plasma, but does inhibit growth of Ph+ leukemia cells in murine CNS which express wt and mutated BCR-ABL (Yokota A et al (2007) Blood 109:306-314).

There is a need for accurate, quantitative prognostic and predictive factors that can assist the practicing physician to make intelligent treatment choices, adapted to a particular patient's needs and cellular physiology and genetic character of their cancer. Also, it is well established that differential expression and activity of proteins in signaling and cellular response pathways can have significant and clinically relevant effects on response and sensitivity to cancer agents, even targeted cancer agents. Various genes and their proteins feed into complex cellular pathways, any significant alteration of which can result in altered prognosis and therapeutic response(s). There exists a need to further identify and particularly characterize these cancer effectors and tumor suppressors, including PTEN, and alterations in PTEN expression and/or activity via post translational or other means that can ultimately be used in monitoring, evaluating, diagnosing, and therapeutic design strategies for the improved treatment and outcome in cancer.

The citation of references herein shall not be construed as an admission that such is prior art to the present invention.

### SUMMARY OF THE INVENTION

In a general aspect, the present invention relates to posttranslational modifications of proteins involved in the cancer pathway and the resultant altered sensitivity to cancer therapeutic modalities and prognosis of cancer. In particular, PTEN phosphorylation, particularly phosphorylation of Y240 in PTEN, has been identified and correlated with altered sensitivity or resistance to targeted cancer therapies, such as kinase receptor inhibitors including EGFR inhibitors, and a poorer prognosis or reduced survival time from diagnosis.

In accordance with the present invention post-translational phosphorylation of PTEN, particularly tyrosine 240 (Y240) has been identified, phosphospecific antibodies generated thereto, and the phosphorylation thereof has been correlated with poorer prognosis in cancer patients and also an increased resistance and decreased/altered sensitivity to cancer agents.

The cellular resistance to targeted therapy can be driven by src-family kinase-mediated PTEN phosphorylation.

In an aspect, phosphorylation of Y240 in PTEN is correlated with non-responsiveness to tyrosine kinase inhibitors, including EGFR inhibitors, in cells harboring this post-translational modification. Y240 phosphorylation on PTEN modulates EGFR signaling, particularly EGFR vIII signaling, and the cellular response to EGFR inhibitors, including Erlotinib. In a particular aspect, glioblastoma cells showing Y240 PTEN phosphorylation demonstrate altered sensitivity to EGFR inhibitors. In a further aspect, PTEN Y240 phosphorylation is correlated with both upfront and acquired resistance to inhibitors, including EGFR inhibitors in glioblastoma (GBM).

In accordance with the above, blocking or reduction of PTEN phosphorylation may be utilized to sensitize tumors or cancer cells to inhibition, including but not limited to EGFR inhibition. In an aspect thereof, blocking PTEN phosphorylation by suppression of src-family kinase activity, can be used to sensitize or resensitize tumors to cancer therapy, in an aspect thereof to sensitize tyrosine kinase receptor-mediated cancers to tyrosine kinase inhibitors. In one such aspect, blocking PTEN phosphorylation by suppression of src-family kinase activity, particularly of lyn activity, can be used to sensitize or resensitize tumors to cancer therapy, in an aspect thereof to sensitize ErbB-mediated cancers to ErbB family inhibitors. In one such aspect, blocking PTEN phosphorylation by suppression of src-family kinase activity, particularly of lyn activity, can be used to sensitize or resensitize tumors to cancer therapy, in an aspect thereof to sensitize EGFR-mediated cancers to EGFR inhibitors. The phosphorylation of PTEN alters or blocks PTEN activity, which leads to downstream effects and aberrant activation of PI3K/AKT signaling. Altered PI3K/AKT signaling is well recognized to be correlated with cancer, particularly aggressive or resistant cancers.

PTEN is now shown to be susceptible to src-family kinase phosphorylation at a total of 6 tyrosine residues, four tyrosines (Y46, Y68, Y155 and Y174) in the phosphatase domain of PTEN, and two tyrosines (Y240 and Y315) in the PTEN C2 domain. Phosphospecific antibodies have been generated and phosphorylation of Y240 is now shown to be correlated with failure to respond to targeted inhibitors, including EGFR inhibitors such as Erlotinib or Gefitinib. Phosphorylated peptide FMY[243.03]FEFPQPLPVC[160.03]GDIK (SEQ ID NO:1) competes away antibody staining in immunostaining techniques. Elevated or comparatively high src-family kinase (SFK) phosphorylation is additionally correlated with failure to respond or comparatively poor response to therapy.

The invention thus provides a method for determining sensitivity or resistance to a therapeutic agent in a cancer patient comprising detecting tyrosine phosphorylation of PTEN in a tumor biopsy or cancer cell sample. In an aspect of the method, Y240 PTEN phosphorylation is determined and the presence of Y240 phosphorylation indicates reduced sensitivity or resistance to a therapeutic agent. In a further aspect, phosphorylation of PTEN is determined by binding or recognition of a phosphospecific antibody.

The invention provides a method for determining sensitivity or resistance to a therapeutic agent in a cancer patient comprising detecting tyrosine phosphorylation of PTEN in a tumor biopsy or cancer cell sample wherein phosphorylation of PTEN is determined at Y240 of PTEN by binding or recognition of a phosphospecific antibody which specifically recognizes tyrosine 240 (Y240) specific phosphorylation of PTEN.

In an aspect of the method(s) of the invention, the therapeutic agent is a tyrosine kinase receptor inhibitor and the presence of Y240 phosphorylation indicates reduced sensitivity or resistance to said inhibitor. In an additional aspect of the method(s) of the invention, the therapeutic agent is an ErbB tyrosine kinase receptor inhibitor and the presence of Y240 phosphorylation indicates reduced sensitivity or resistance to said ErbB inhibitor. In a further aspect of the method(s) of the invention, the therapeutic agent is an EGFR kinase inhibitor and the presence of Y240 phosphorylation indicates reduced sensitivity or resistance to said EGFR inhibitor.

In an additional aspect, a method is included wherein the therapeutic agent is a src-family kinase inhibitor and the presence of PTEN phosphorylation indicates sensitivity to said src-family kinase inhibitor. In one such method, the therapeutic agent is a src-family kinase lyn inhibitor and the presence of phosphorylation at Y240 of PTEN indicates sensitivity to said src-family kinase lyn inhibitor.

The invention includes a method for determining sensitivity or resistance to an EGFR inhibitor in a cancer patient comprising detecting tyrosine phosphorylation of PTEN in a tumor biopsy or cancer cell sample. In an aspect thereof, phosphorylation of PTEN is determined by binding or recognition a phosphospecific antibody. In a further particular such method, phosphorylation of PTEN is determined at Y240 of PTEN by binding or recognition of a phosphospecific antibody which specifically recognizes tyrosine 240 (Y240) specific phosphorylation of PTEN.

The methods of the invention have use and/or application in cancer, including wherein cancer is selected from brain, melanoma, breast, prostate, renal, endometrial, lung, stomach, colon, gastrointestinal tract, and kidney cancer. The relevant cancer may be brain cancer, particularly astrocytoma. In a particular aspect, the cancer is glioblastoma.

A method is further provided for determining the prognosis of cancer in a mammal comprising detecting tyrosine 240 phosphorylation of PTEN in a tumor biopsy or cancer cell sample from said mammal, wherein Y240 PTEN phosphorylation is indicative of a poor prognosis or reduced survival time on cancer diagnosis in said mammal. In an aspect thereof, the cancer is a tyrosine kinase receptor-mediated cancer. In an aspect thereof, the cancer is an ErbB-mediated cancer. In a further aspect thereof, the cancer is an EGFR-mediated cancer.

The invention includes a method for determining the prognosis of cancer in a mammal comprising detecting tyrosine 240 phosphorylation of PTEN in a tumor biopsy or cancer cell sample from said mammal and further comprising detecting EGFR expression and/or determining the presence of EGFR vIII, wherein Y240 PTEN phosphorylation, combined with increased EGFR expression and the presence of EGFR vIII is indicative of a poor prognosis or reduced survival time on cancer diagnosis in said mammal.

The cancer for prognosis and/or monitoring includes any cancer wherein PTEN expression and/or activity is relevant. The cancer may be selected from brain, melanoma, breast, prostate, renal, endometrial, lung, stomach, colon, and kidney. In an aspect, the brain cancer and is astrocytoma. In a particular such aspect, the cancer is glioblastoma.

In an aspect of the invention a method of monitoring and treating cancer in a mammal is provided comprising:

(a) detecting tyrosine phosphorylation of PTEN in a tumor biopsy or cancer cell sample of said mammal and determining whether Y240 of PTEN is phosphorylated;

(b) administering a src-family kinase inhibitor alone or in combination with one or more other cancer therapeutic agent to any said mammal wherein Y240 of PTEN is phosphorylated; and

(c) optionally monitoring Y240 PTEN phosphorylation in said mammal after administration of the inhibitor.

In a particular aspect of the method, phosphorylation at Y240 of PTEN is determined by binding or recognition of a phosphospecific antibody which specifically recognizes tyrosine 240 (Y240) specific phosphorylation of PTEN.

The present invention includes an assay system which may be prepared in the form of a test kit for the quantitative analysis of the extent of the presence of, for instance, phosphorylated PTEN, particularly Y240 phosphorylated PTEN. The system or test kit may comprise a labeled component prepared by one of the radioactive and/or enzymatic techniques discussed herein, coupling a label to the antibody, and one or more additional immunochemical reagents, at least one of which is a free or immobilized components to be determined or their binding partner(s).

The present invention includes a kit for therapeutic monitoring or prognosis of cancer in an EGFR-mediated cancer, said kit comprising a phosphospecific anti-PTEN antibody or fragment thereof which recognizes tyrosine 240 (Y240) specific phosphorylation of PTEN and does not react with PTEN which lacks phosphorylation at Y240, optionally with reagents and/or instructions for use.

Specific antibodies, particularly phosphospecific antibodies, are provided and are a relevant aspect of the invention. The invention thus provides and includes a phosphospecific antibody molecule or fragment thereof which recognizes tyrosine 240 (Y240) specific phosphorylation of PTEN and does not react with PTEN which lacks phosphorylation at Y240. The antibody may be a polyclonal antibody. In an aspect, the specific antibody is a monoclonal antibody. Fragments, particularly specific binding fragments, of the antibody(ies) are contemplated in the invention.

The unique specificity and affinity of the antibodies and fragments of the invention to particular phosphorylated protein provides diagnostic and therapeutic uses to identify, characterize and target post-translationally modified PTEN and its correlation with drug sensitivity and cancer prognosis. Cancers whose drug sensitivity, progression rate, and cell signaling are particularly susceptible to PTEN phosphorylation and changes in PTEN activity may be targeted, evaluated and monitored by the phosphorylation specific antibodies of the present invention. Such cancers include brain, melanoma, breast, prostate, renal, endometrial, lung, stomach, colon, and kidney cancer. Particular cancers include astrocytomas, particularly glioblastomas.

The diagnostic utility of the present invention extends to the use of the antibodies of the present invention in assays to characterize tumors or cellular samples or to screen for tumors or cancer, including in vitro and in vivo diagnostic assays. In an immunoassay, a control quantity of the antibodies, peptides, or the like may be prepared and labeled with an enzyme, a specific binding partner and/or a radioactive element, and may then be introduced into a cellular sample. After the labeled material or its binding partner(s) has had an opportunity to react with sites within the sample, the resulting mass may be examined by known techniques, which may vary with the nature of the label attached.

Antibodies or peptides of the invention may carry a detectable or functional label. The antibodies or peptides, or their binding partners, may carry a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, 121I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc and ¹⁸⁶Re. When radioactive labels are used, known currently available counting procedures may be utilized to identify and quantitate the specific binding members. In the instance where the label is an enzyme, detection may be accomplished by any of the presently utilized colorimetric, spectrophotometric, fluorospectrophotometric, amperometric or gasometric techniques known in the art.

The radiolabelled antibodies and fragments thereof or peptides, are useful in in vitro diagnostics techniques and in in vivo radioimaging techniques. In a further aspect of the invention, radiolabelled antibodies and fragments thereof, particularly radioimmunoconjugates, are useful in radioimmunotherapy, particularly as radiolabelled antibodies for cancer therapy. In a still further aspect, the radiolabelled specific binding members, particularly antibodies and fragments thereof, are useful in radioimmuno-guided surgery techniques, wherein they can identify and indicate the presence and/or location of cancer cells, precancerous cells, tumor cells, and hyperproliferative cells, prior to, during or following surgery to remove such cells.

Immunoconjugates or antibody fusion proteins of the present invention, wherein the antibodies and fragments thereof of the present invention are conjugated or attached to other molecules or agents further include, but are not limited to binding members conjugated to a chemical ablation agent, toxin, immunomodulator, cytokine, cytotoxic agent, chemotherapeutic agent or drug.

The present invention also includes antibodies and fragments thereof, which are covalently attached to or otherwise associated with other molecules or agents. These other molecules or agents include, but are not limited to, molecules (including antibodies or antibody fragments) with distinct recognition characteristics, toxins, ligands, and chemotherapeutic agents. In an additional aspect the antibodies or fragments of the invention may be used to target or direct therapeutic molecules or other agents, for example to target molecules or agents to Y240 phosphorylated PTEN expressing cells, for example to such cells in cancerous lesions.

In a further embodiment, the present invention relates to certain therapeutic methods which would be based upon the presence and/or level of phosphorylated PTEN, particularly Y240 PTEN, or upon agents or other drugs determined to alter the presence and/or level of phosphorylated PTEN, particularly Y240 PTEN. A first therapeutic method is associated with the prevention or treatment of cancer, including but not limited to brain, melanoma, breast, prostate, renal, endometrial, lung, stomach, colon, and kidney cancer. In an aspect, the brain cancer is astrocytoma. In a particular such aspect, the cancer is glioblastoma. In one such aspect, assays to determine the presence and/or amount of phosphorylated PTEN, particularly Y240 PTEN, alone or in combination with other cancer markers (such as EGFR, PI3K/AKT, Src-family kinase(s) (Src, Lyn), P53 etc), are utilized as part of the initial cancer diagnostic procedure, following initial diagnosis, during therapy, and/or post therapy to determine and monitor phosphorylated PTEN, particularly pY240 PTEN. The phosphorylation status of PTEN is utilized to determine initial cancer therapy and targeted inhibitors, as well as during treatment to monitor sensitivity and/or resistance to current therapy and determine the best mode of initial and continued therapy. In an aspect, the phosphorylation status of PTEN is utilized to determine and assess prognosis of a cancer patient, including on initial diagnosis.

Thus, methods are provided for treatment of tyrosine kinase receptor mediated cancers, including ErbB-mediated cancers and EGFR-mediated cancers, whereby the presence and/or amount of phosphorylated PTEN, particularly Y240 PTEN, alone or in combination with other cancer markers, is determined in a cancer patient and the patient is treated with an ErbB or EGFR inhibitor, alone, or in combination with an additional kinase inhibitor, such as a Src and/or Lyn kinase inhibitor. In a treatment of the invention, receptor tyrosine kinase inhibitors are combined with modulators of PTEN phosphorylation, such as a Src or Lyn kinase inhibitor, or with modulators of the phosphorylated PTEN signaling process, such as PI3K/AKT inhibitors.

In an aspect of the invention, a method is provided for inducing sensitivity of a cancer to an ErbB inhibitor where said cancer had obtained resistance to the ErbB inhibitor by Y240 phosphorylation of PTEN, comprising administering a combination of a modulator that blocks phosphorylation of PTEN or blocks the downstream signal which results from PTEN phosphorylation. In an aspect of the invention, a method is provided for inducing sensitivity of a cancer to an EGFR inhibitor where said cancer had obtained resistance to the EGFR inhibitor by Y240 phosphorylation of PTEN, comprising administering a combination of a modulator that blocks phosphorylation of PTEN or blocks the downstream signal which results from PTEN phosphorylation.

The invention thus provides a method for inducing sensitivity of a cancer to an ErbB inhibitor where said cancer had obtained resistance to the ErbB inhibitor by Y240 phosphorylation of PTEN, comprising administering a combination of a modulator that blocks phosphorylation of PTEN. The invention provides a method for inducing sensitivity of a cancer to an EGFR inhibitor where said cancer had obtained resistance to the EGFR inhibitor by Y240 phosphorylation of PTEN, comprising administering a combination of a modulator that blocks phosphorylation of PTEN. In an aspect of these methods, the modulator that blocks phosphorylation of PTEN is a Src kinase inhibitor. In a further aspect, the modulator is a Lyn kinase inhibitor.

The invention includes a method of treating an ErbB family phospho Y240 PTEN positive tumor in a mammal comprising administering a src family targeted tyrosine kinase inhibitor, followed by or in combination with treatment with an ErbB family targeted tyrosine kinase inhibitor. In an aspect of this method a method is provided for treating an EGFR overexpressing or mutant phospho Y240 PTEN positive tumor in a mammal comprising administering a src family targeted tyrosine kinase inhibitor, followed by or in combination with treatment with an EGFR targeted tyrosine kinase inhibitor. In an aspect of these methods the src family targeted tyrosine kinase inhibitor is Dasatinib or INNO-406.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

FIG. 1 depicts detection of tyrosine phosphorylation on PTEN in astrocytes and GBM cells. A, 293T cells were co-transfected with FLAG-HA-PTEN constructs and V5-tagged c-src. Phosphorylation of Y240 was detected by immunoblotting of anti-HA immunoprecipitates with a site-specific polyclonal antibody. B, PTEN^{loxP/loxP}/Ink4a/arf^{-/-} astrocytes (see text) expressing wild-type (lanes 2 and 4) or constitutively active c-src (lanes 3 and 5) were serum-starved for 48 hours prior to IP of PTEN with a goat polyclonal antibody. PTEN phosphorylation was detected by immunoblotting with anti-phosphotyrosine (4G10). C, Ink4a/Arf/PTEN^{-/-} astrocytes reconstituted with wild type or Y240/315F PTEN alleles were serum-starved prior to pervanadate treatment as indicated. PTEN immunoprecipitates were immunoblotted with anti-phosphotyrosine (4G10). D, Western blots of GBM39 cells and TS543 cells to assess pY240 PTEN and PTEN levels (PY416 SFK as control in TS543 cells). GBM39 cells are positive for EGFR vIII and PTEN. GBM39 cells were grown in the presence or absence of pervanadate 100 μM (which inhibits tyrosine phosphatases) and/or dasatinib (100 nM) (a src kinase inhibitor). The TS543 neurosphere line, which is PTEN and PDGFR positive, was exposed to 100 nM dasatinib for 24 hours as indicated.

FIG. 2 depicts PTEN and SFK phosphorylation in human GBMs that failed to respond to EGFR inhibitor therapy. A, correlation between immunohistochemical staining of src-family kinases on pY416 and PTEN staining on pY240 in tissue sections from human GBMs, both pre- and post-EGFR inhibitor treatment as indicate^{d. B, PTE}N pY240 an^{d s}rc pY416 staining are significantly elevated in tumors of EGFR inhibitor non-responders (examples of typical p-src and p-PTEN staining patterns for responder and non-responder tumors shown). C, Immunohistochemical analysis of the tumors of two patients who initially responded to therapy but at ^{ti}me of treatment failure, displayed increased p-src and p-PTEN (actual staining shown for one such patient).

FIG. 3 shows that PTEN Y240F displays enhanced inhibition of EGFRvIII signaling. A, U87MG cells expressing WT or Y240F PTEN were fixed and stained with propidium iodide for analysis of DNA content by flow cytometry. Data shown is representative of at least three independent experiments. B, Ink4a/Arf/PTEN^{-/-} astrocytes expressing active c-src where indicated were reconstituted with WT or Y240F PTEN alleles and PTEN function was assessed by immunoblotting for the phosphorylation of AKT on S473. C, Lysates from U87-EGFRvIII cells expressing WT or Y240F PTEN alleles were analyzed for the activation of downstream signaling pathways by blotting with phospho-specific antibodies. Phosphorylation of WT PTEN on Y240 was detected by anti-pY240 IP followed by blotting with anti-PTEN (bottom panel).

FIG. 4 depicts that manipulation of src activity and phosphorylation of PTEN on Y240 modulates the cellular response to Erlotinib. A, Ink4A/Arf^{-/-} astrocytes were exposed to increasing doses of Erlotinib for 72 hours in the presence or absence of Dasatinib as indicated. Cell viability was measured by WST-1 assay. B, Ink4a/Arf^{-/-} astrocytes expressing EGFRvIII were treated as in (a). C, Ink4a/Arf/PTEN^{-/-} expressing EGFRvIII were reconstituted with WT or Y240F PTEN alleles were exposed to Erlotinib for 72 hours prior to measurement of cell viability by WST-1 assay. D, Correlation between p-EGFR (Y1086) and p-PTEN (Y240) immunohistochemical staining in human clinical samples pre- and post-treatment with EGFR inhibitors as indicated.

FIG. 5 provides demonstration of anti-pY240 specificity in immunohistochemistry by peptide competition. Tissue sections from human GBM samples known to express wild type PTEN were stained with anti-p Y240 in the absence or presence of either the phospho-peptide used to generate the antibody (see methods) or the non-phosphorylated version of the peptide.

FIG. 6: In GBM tumors showing loss of PTEN, pY240 staining is restricted to regions in which PTEN expression has been retained. Adjacent sections from a human GBM sample showing areas of PTEN loss were stained with either anti-PTEN or anti-pY240 (PTEN).

FIG. 7: PTEN WT and PTEN Y240F both induce G1 arrest when expressed in U87MG cells. U87MG cells expressing WT or Y240F PTEN were fixed and stained with propidium iodide for analysis of DNA content by flow cytometry. Data shown is representative of at least three independent experiments.

FIG. 8 depicts expression of EGFRvIII sensitizes astrocytes to Erlotinib. Ink4a/Are^{-/-} astrocytes engineered to express EGFRvIII where indicated were grown for 72 hours in the presence of increasing concentrations of Erlotinib before measurement of cell viability using the WST-1 assay.

FIG. 9: Model for modulation of the response to EGFR inhibitors by PTEN phosphorylation. Hypophosphorylated PTEN (top) is able to effectively inhibit signaling from EGFRvIII both through PI3K/AKT through other downstream targets, by regulating receptor levels. In tumors with high src activity, PTEN becomes phosphorylated ad inhibited, thus EGFRvIII levels increase and a higher dose of EGFR inhibitor is required to achieve effective inhibition of signaling.

FIG. 10 graphically depicts cumulative survival of glioblastoma patients that are positive for EGFR hyperexpression and PTEN expression, comparing those that are also positive for PTEN phosphorylation and those that are negative for PTEN phosphorylation.

FIG. 11 graphically depicts cumulative survival of glioblastoma patients that are positive for EGFR mutant vIII and PTEN expression, comparing those that are also positive for PTEN phosphorylation and those that are negative for PTEN phosphorylation.

FIG. 12 provides a spreadsheet of data results of all astrocytoma and glioblastoma cancer patients studied, indicating diagnosis, EGFR characteristics (expression, amplification, vIII status, and IHC score), PTEN characteristics (score of total PTEN, score of Y240 phosphorylation), p53 status (mutation, IHC score), and survival (alive or dead status, cumulative survival time in months)

Expression of PTEN total and phosphorylated by immunohistochemistry (IHC) score is tabulated in FIG. 13, with PTEN scores of 0-4 graphed versus number of astrocytoma patients. Number of PTEN positive and negative patients at diagnosis are displayed in FIG. 14.

FIG. 13 graphically presents number of patients versus PTEN (total and phosphorylated) by immunohistochemistry (IHC) score (0-4).

FIG. 14 graphically presents PTEN status (total negative or total positive) of versus number of patients.

FIG. 15 provides Western blot studies of total PTEN and phosphorylated Y240 PTEN upon cotransfection of 293 cells with either src or lyn in combination with wild type (WT) PTEN or PTEN Y240F, which cannot be phosphorylated. 4G10 phosphotryrosine is a control phosphorylated protein.

FIG. 16 depicts Western blots for pY240 PTEN and Total PTEN in soluble and pellet fractions of RIPA lysates from 293 cells cotransfected with Lyn and either wild type PTEN or Y240F PTEN mutant, which cannot be phosphorylated at amino acid 240 due to the substitution of phenylalanine for tyrosine at amino acid 240.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

### A. TERMINOLOGY

The term “antibody” describes an immunoglobulin whether natural or partly or wholly synthetically produced. The term also covers any polypeptide or protein having a binding domain which is, or is homologous to, an antibody binding domain. CDR grafted antibodies are also contemplated by this term. An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567. The term “antibody(ies)” includes a wild type immunoglobulin (Ig) molecule, generally comprising four full length polypeptide chains, two heavy (H) chains and two light (L) chains, or an equivalent Ig homologue thereof (e.g., a camelid nanobody, which comprises only a heavy chain); including full length functional mutants, variants, or derivatives thereof, which retain the essential epitope binding features of an Ig molecule, and including dual specific, bispecific, multispecific, and dual variable domain antibodies; Immunoglobulin molecules can be of any class (e.g., IgG, IgE, IgM, IgD, IgA, and IgY), or subclass (e.g., IgG1, IgG2, IgG3, IgG4, IgA1, and IgA2). Also included within the meaning of the term “antibody” are any “antibody fragment”.

An “antibody fragment” means a molecule comprising at least one polypeptide chain that is not full length, including (i) a Fab fragment, which is a monovalent fragment consisting of the variable light (VL), variable heavy (VH), constant light (CL) and constant heavy 1 (CH1) domains; (ii) a F(ab′)2 fragment, which is a bivalent fragment comprising two Fab fragments linked by a disulfide bridge at the hinge region; (iii) a heavy chain portion of an Fab (Fd) fragment, which consists of the VH and CH1 domains; (iv) a variable fragment (Fv) fragment, which consists of the VL and VH domains of a single arm of an antibody, (v) a domain antibody (dAb) fragment, which comprises a single variable domain (Ward, E. S. et al., Nature 341, 544-546 (1989)); (vi) a camelid antibody; (vii) an isolated complementarity determining region (CDR); (viii) a Single Chain Fv Fragment wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (ix) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites (WO94/13804; P. Holliger et al Proc. Natl. Acad. Sci. USA 90 6444-6448, (1993)); and (x) a linear antibody, which comprises a pair of tandem Fv segments (VH-CH1-VH-CH1) which, together with complementarity light chain polypeptides, form a pair of antigen binding regions; (xi) multivalent antibody fragments (scFv dimers, trimers and/or tetramers (Power and Hudson, J Immunol. Methods 242: 193-204 9 (2000)); and (xii) other non-full length portions of heavy and/or light chains, or mutants, variants, or derivatives thereof, alone or in any combination.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023 and U.S. Pat. Nos. 4,816,397 and 4,816,567.

An “antibody combining site” is that structural portion of an antibody molecule comprised of light chain or heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Antibodies may also be bispecific, wherein one binding domain of the antibody is a specific binding member of the invention, and the other binding domain has a different specificity, e.g. to recruit an effector function or the like. Bispecific antibodies of the present invention include wherein one binding domain of the antibody is a specific binding member of the present invention, including a fragment thereof, and the other binding domain is a distinct antibody or fragment thereof, including that of a distinct anti-cancer or anti-tumor specific antibody. The other binding domain may be an antibody that recognizes or targets a particular cell type, as in a neural or glial cell-specific antibody. In the bispecific antibodies of the present invention the one binding domain of the antibody of the invention may be combined with other binding domains or molecules which recognize particular cell receptors and/or modulate cells in a particular fashion, as for instance an immune modulator (e.g., interleukin(s)), a growth modulator or cytokine (e.g. tumor necrosis factor (TNF), and particularly, the TNF bispecific modality demonstrated in U.S. Ser. No. 60/355,838 filed Feb. 13, 2002 incorporated herein in its entirety) or a toxin (e.g., ricin) or anti-mitotic or apoptotic agent or factor. Thus, the anti-FAP antibodies of the invention may be utilized to direct or target agents, labels, other molecules or compounds or antibodies to stromal sites, particular areas of wound healing, inflammation, cancer or tumors.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may also contain an antibody molecule having a plurality of antibody combining sites, each immunospecific for a different antigen; e.g., a bispecific (chimeric) monoclonal antibody.

The term “antigen binding domain” describes the part of an antibody which comprises the area which specifically binds to and is complementary to part or all of an antigen. Where an antigen is large, an antibody may bind to a particular part of the antigen only, which part is termed an epitope. An antigen binding domain may be provided by one or more antibody variable domains. Preferably, an antigen binding domain comprises an antibody light chain variable region (VL) and an antibody heavy chain variable region (VH).

Immunoconjugates or antibody fusion proteins of the present invention, wherein the antibodies, antibody molecules, or fragments thereof, of use in the present invention are conjugated or attached to other molecules or agents further include, but are not limited to such antibodies, molecules, or fragments conjugated to a chemical ablation agent, toxin, immunomodulator, cytokine, cytotoxic agent, chemotherapeutic agent, antimicrobial agent or peptide, cell wall and/or cell membrane disrupter, or drug.

The term “specific” may be used to refer to the situation in which one member of a specific binding pair will not show any significant binding to molecules other than its specific binding partner(s). The term is also applicable where e.g. an antigen binding domain is specific for a particular epitope which is carried by a number of antigens, in which case the specific binding member carrying the antigen binding domain will be able to bind to the various antigens carrying the epitope.

The term “comprise” generally used in the sense of include, that is to say permitting the presence of one or more features or components.

The term “consisting essentially of” refers to a product, particularly a peptide sequence, of a defined number of residues which is not covalently attached to a larger product. In the case of the peptide of the invention referred to above, those of skill in the art will appreciate that minor modifications to the N- or C-terminal of the peptide may however be contemplated, such as the chemical modification of the terminal to add a protecting group or the like, e.g. the amidation of the C-terminus.

The term “isolated” refers to the state in which specific binding members of the invention, or nucleic acid encoding such binding members will be, in accordance with the present invention. Members and nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practised in vitro or in vivo. Members and nucleic acid may be formulated with diluents or adjuvants and still for practical purposes be isolated—for example the members will normally be mixed with gelatin or other carriers if used to coat microtitre plates for use in immunoassays, or will be mixed with pharmaceutically acceptable carriers or diluents when used in diagnosis or therapy.

As used herein, “pg” means picogram, “ng” means nanogram, “ug” or “μg” mean microgram, “mg” means milligram, “ul” or “μl” mean microliter, “ml” means milliliter, “l” means liter.

The terms “antibody”, “phosphospecific antibody”, “anti Y240 antibody”, “Y240 PTEN antibody”, “Y240 antibody”, “pY240 antibody”, “PTEN phosphospecific antibody”, “PTEN Y240 phosphospecific antibody” and any variants not specifically listed, may be used herein interchangeably, and as used throughout the present application and claims refer to proteinaceous material including single or multiple proteins, and extends to those proteins having the profile of activities described herein and capable of binding to or recognizing phosphorylated Y240 PTEN and/or phosphorylated PTEN. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Also, the terms “antibody”, “phosphospecific antibody”, “anti Y240 antibody”, “Y240 PTEN antibody”, “Y240 antibody”, “pY240 antibody”, “PTEN phosphospecific antibody”, “PTEN Y240 phosphospecific antibody” are intended to include within their scope proteins specifically recited herein as well as all substantially homologous analogs and allelic variations.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC or CUA or |
| | CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG or AGU or |
| | AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG or AGA or |
| | AGG |
| Glycine (Gly or G) | GGU or GGC or GGA or GGG |
| Tryptophan (Trp or W) | UGG |
| Termination codon | UAA (ochre) or UAG (amber) or UGA (opal) |

It should be understood that the codons specified above are for RNA sequences. The corresponding codons for DNA have a T substituted for U.

Mutations can be made in the sequences encoding the amino acids, peptides, antibody(ies), antibody fragments, such that a particular codon is changed to a codon which codes for a different amino acid. Such a mutation is generally made by making the fewest nucleotide changes possible. A substitution mutation of this sort can be made to change an amino acid in the resulting protein in a non-conservative manner (for example, by changing the codon from an amino acid belonging to a grouping of amino acids having a particular size or characteristic to an amino acid belonging to another grouping) or in a conservative manner (for example, by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

The following is one example of various groupings of amino acids:

Amino Acids with Nonpolar R Groups

### Alanine, Valine, Leucine, Isoleucine, Proline, Phenylalanine, Tryptophan, Methionine

Amino Acids with Uncharged Polar R Groups

### Glycine, Serine, Threonine, Cysteine, Tyrosine, Asparagine, Glutamine

Amino Acids with Charged Polar R Groups (Negatively Charged at Ph 6.0)
Aspartic acid, Glutamic acid

### Basic Amino Acids (Positively Charged at pH 6.0)

### Lysine, Arginine, Histidine (at pH 6.0)

Another grouping may be those amino acids with phenyl groups:

### Phenylalanine, Tryptophan, Tyrosine

Another grouping may be according to molecular weight (i.e., size of R groups):

| | | | |
|---|---|---|---|
| Glycine | 75 | Alanine | 89 |
| Serine | 105 | Proline | 115 |
| Valine | 117 | Threonine | 119 |
| Cysteine | 121 | Leucine | 131 |
| Isoleucine | 131 | Asparagine | 132 |
| Aspartic acid | 133 | Glutamine | 146 |
| Lysine | 146 | Glutamic acid | 147 |
| Methionine | 149 | Histidine (at pH 6.0) | 155 |
| Phenylalanine | 165 | Arginine | 174 |
| Tyrosine | 181 | Tryptophan | 204 |

Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

Exemplary and preferred conservative amino acid substitutions include any of: glutamine (Q) for glutamic acid (E) and vice versa; leucine (L) for valine (V) and vice versa; serine (S) for threonine (T) and vice versa; isoleucine (I) for valine (V) and vice versa; lysine (K) for glutamine (Q) and vice versa; isoleucine (I) for methionine (M) and vice versa; serine (S) for asparagine (N) and vice versa; leucine (L) for methionine (M) and vice versa; lysine (L) for glutamic acid (E) and vice versa; alanine (A) for serine (S) and vice versa; tyrosine (Y) for phenylalanine (F) and vice versa; glutamic acid (E) for aspartic acid (D) and vice versa; leucine (L) for isoleucine (I) and vice versa; lysine (K) for arginine (R) and vice versa.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

Two amino acid sequences are “substantially homologous” when at least about 70% of the amino acid residues (preferably at least about 80%, and most preferably at least about 90 or 95%) are identical, or represent conservative substitutions. The CDR regions of two antibodies are substantially homologous when one or mre amino acids are substituted with a similar or conservative amino acid substitution, and wherein the antibody/antibodies have the profile of binding and activities of one or more of ESC11 or ESC14 disclosed herein.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

The term ‘agent’ means any molecule, including polypeptides, antibodies, polynucleotides, chemical compounds and small molecules. In particular the term agent includes compounds such as test compounds or drug candidate compounds.

The term ‘agonist’ refers to a ligand that stimulates the receptor the ligand binds to in the broadest sense.

The term ‘assay’ means any process used to measure a specific property of a compound. A ‘screening assay’ means a process used to characterize or select compounds based upon their activity from a collection of compounds.

The term ‘preventing’ or ‘prevention’ refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset.

The term ‘prophylaxis is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Therapeutically effective amount’ means that amount of a drug, compound, antimicrobial, antibody, or pharmaceutical agent that will elicit the biological or medical response of a subject that is being sought by a medical doctor or other clinician. In particular, with regard to gram-positive bacterial infections and growth of gram-positive bacteria, the term “effective amount” is intended to include an effective amount of a compound or agent that will bring about a biologically meaningful decrease in the amount of or extent of infection of gram-positive bacteria, including having a bacteriocidal and/or bacteriostatic effect. The phrase “therapeutically effective amount” is used herein to mean an amount sufficient to prevent, and preferably reduce by at least about 30 percent, more preferably by at least 50 percent, most preferably by at least 90 percent, a clinically significant change in the growth or amount of infectious bacteria, or other feature of pathology such as for example, elevated fever or white cell count as may attend its presence and activity.

The term ‘treating’ or ‘treatment’ of any disease or infection refers, in one embodiment, to ameliorating the disease or infection (i.e., arresting the disease or growth of the infectious agent or bacteria or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or infection, either physically, (e.g., stabilization of a discernible symptom), physiologically, (e.g., stabilization of a physical parameter), or both. In a further embodiment, ‘treating’ or ‘treatment’ relates to slowing the progression of a disease or reducing an infection.

The phrase pharmaceutically acceptable” refers to molecular entities and compositions that are physiologically tolerable and do not typically produce an allergic or similar untoward reaction, such as gastric upset, dizziness and the like, when administered to a human.

As used herein, “pg” means picogram, “ng” means nanogram, “ug” or “μg” mean microgram, “mg” means milligram, “ul” or “μl” mean microliter, “ml” means milliliter, “l” means liter.

### B. DETAILED DISCLOSURE

In a general aspect, the present invention relates to posttranslational modifications of proteins involved in the cancer pathway and the resultant altered sensitivity to cancer therapeutic modalities and prognosis of cancer. In particular, PTEN phosphorylation, particularly phosphorylation of Y240 in PTEN, has been identified and correlated with altered sensitivity or resistance to targeted cancer therapies, such as EGFR inhibitors, and a poorer prognosis or reduced survival time from diagnosis. Post-translational phosphorylation of PTEN, particularly tyrosine 240 (Y240) has been identified, phosphospecific antibodies generated thereto, and the phosphorylation thereof has been correlated with poorer prognosis in cancer patients and also an increased resistance and decreased/altered sensitivity to cancer agents.

In accordance with the present invention, phosphorylation of Y240 in PTEN is correlated with non-responsiveness to EGFR inhibitors in cells harboring this post-translational modification. Y240 phosphorylation on PTEN modulates EGFR signaling, particularly EGFR vIII signaling, and the cellular response to EGFR inhibitors, including Erlotinib. In a particular aspect, glioblastoma cells showing Y240 PTEN phosphorylation demonstrate altered sensitivity to EGFR inhibitors. PTEN Y240 phosphorylation is correlated with both upfront and acquired resistance to inhibitors, including EGFR inhibitors in glioblastoma (GBM).

In a further application and aspect of the invention, blocking or reduction of PTEN phosphorylation may be utilized to sensitize tumors or cancer cells to inhibition, including but not limited to EGFR inhibition. In one such aspect, blocking PTEN phosphorylation by suppression of src-family kinase activity, particularly of lyn activity, can be used to sensitize or resensitize tumors to cancer therapy, in an aspect thereof to sensitize EGFR-mediated cancers to EGFR inhibitors. The phosphorylation of PTEN alters or blocks PTEN activity, which leads to downstream effects abd aberrant activation of PI3K/AKT signaling. Altered PI3K/AKT signaling is well recognized to be correlated with cancer, particularly aggressive or resistant cancers.

PTEN is now shown to be susceptible to src-family kinase phosphorylation at a total of 6 tyrosine residues, four tyrosines (Y46, Y68, Y155 and Y174) in the phosphatase domain of PTEN, and two tyrosines (Y240 and Y315) in the PTEN C2 domain. Phosphospecific antibodies have been generated and phosphorylation of Y240 is now shown to be correlated with failure to respond to targeted inhibitors, including EGFR inhibitors such as Erlotinib or Gefitinib. Phosphorylated peptide FMY[243.03]FEFPQPLPVC[160.03]GDIK competes away antibody staining in immunostaining techniques. Elevated or comparatively high src-family kinase (SFK) phosphorylation is additionally correlated with failure to respond or comparatively poor response to therapy.

The invention thus provides a method for determining sensitivity or resistance to a therapeutic agent in a cancer patient comprising detecting tyrosine phosphorylation of PTEN in a tumor biopsy or cancer cell sample. In an aspect of the method, Y240 PTEN phosphorylation is determined and the presence of Y240 phosphorylation indicates reduced sensitivity or resistance to a therapeutic agent. In a further aspect, phosphorylation of PTEN is determined by binding or recognition of a phosphospecific antibody.

Phosphatase and tensin homolog (PTEN) is a tumor suppressor gene mutated in many human cancers. The major substrate of its lipid phosphatase activity is PIP3. By dephosphorylating PIP3, PTEN negatively regulates the PI3K pathway and AKT activation and thus suppresses tumorigenesis. PI3K is a downstream effector of the EGFR cascade. PTEN is the second most frequently mutated tumor suppressor gene in human sporadic cancer and reduced PTEN expression occurs in approximately half of all tumors (Suzuki, A. et al (2008) Cancer Sci 99(2): 209-213). Human PTEN is a 403 amino acid phosphatase and its sequence has been described and is known (Myers M P et al (1997) PNAS USA 94(17):9052-9057; Maehama T and Dixon J E (1998) J Biol Chem 273(22):13375-13378; Myers M P et al (1998) PNAS USA 95(23):13513-13518; Genbank Accession Numbers P60484, NP_{—}000305.3 and AAH05821.1).

### Diagnostic Assays

The present invention also relates to a variety of diagnostic applications, including methods for determining sensitivity or resistance to a therapeutic agent in a cancer patient, methods for determining the prognosis of cancer, methods for assessing predicted overall survival in a cancer patient, methods of monitoring and treating cancer, any and each wherein tyrosine phosphorylation of PTEN is detected and determined in a tumor biopsy, cellular sample, or cancer cell sample. In an aspect of the methods, Y240 PTEN phosphorylation is determined and the presence of Y240 phosphorylation indicates reduced sensitivity or resistance to a therapeutic agent, correlates with poorer prognosis and reduced overall survival, and the responsiveness to certain inhibitors or treatments is predicted or determined.

Phosphorylation of PTEN may be determined by binding or recognition of a phosphospecific antibody. The binding or recognition of a phosphospecific antibody, particularly a Y240 PTEN phosphospecific antibody, may be competed with a tyrosine 240 mutated peptide, a Y240 phosphorylated PTEN peptide. Phosphorylation of PTEN may be determined in combination with and/or prior or subsequent to, other cancer relevant markers of proteins, for example but not by limitation, EGFR expression, EGFR vIII mutant status, p60 Src, Lyn and/or one or more other Src-family kinase activity, expression and/or phosphorylation, PI3K/AKT, ErbB2, STAT3/4 etc. A determination of the combined status and/or expression of PTEN and other cancer markers contributes to a clinical profile of the therapeutic susceptibility, sensitivity and resistance of a cancer patient, tumor sample, or cell sample.

PTEN phosphorylation is determined in any sample or under any conditions wherein phosphorylation is maintained and inherently stabilized. Thus, the PTEN protein should be retained in any diagnostic applications in its native and post-translationally modified form, and may be particularly maintained in its natural or normal cellular or nuclear location in a cell. Immunohistochemistry, immunoblotting, FACS analysis, cell sorting, tissue sections, etc may be utilized in the methods herein.

The invention provides a method for determining sensitivity or resistance to a therapeutic agent in a cancer patient comprising detecting tyrosine phosphorylation of PTEN in a tumor biopsy or cancer cell sample wherein phosphorylation of PTEN is determined at Y240 of PTEN by binding or recognition of a phosphospecific antibody which specifically recognizes tyrosine 240 (Y240) specific phosphorylation of PTEN. The therapeutic agent may be an EGFR kinase inhibitor and the presence of Y240 phosphorylation may thus indicate reduced sensitivity or resistance to said EGFR inhibitor. The therapeutic agent may be a src-family kinase inhibitor and the presence of PTEN phosphorylation may thus indicate sensitivity to said src-family kinase inhibitor. The therapeutic agent may be a src-family kinase lyn inhibitor and the presence of phosphorylation at Y240 of PTEN thus indicate sensitivity to said src-family kinase lyn inhibitor.

The methods of the invention have use and/or application in cancer, including wherein cancer is selected from brain, melanoma, breast, prostate, renal, endometrial, lung, stomach, colon, and kidney cancer. The relevant cancer may be brain cancer, particularly astrocytoma. In a particular aspect, the cancer is glioblastoma.

A method is further provided for determining the prognosis of cancer in a mammal comprising detecting tyrosine 240 phosphorylation of PTEN in a tumor biopsy or cancer cell sample from said mammal, wherein Y240 PTEN phosphorylation is indicative of a poor prognosis or reduced survival time on cancer diagnosis in said mammal. In an aspect thereof, the cancer is an EGFR-mediated cancer.

Diagnostic applications of the present invention, particularly with regard ro PTEN phosphospecific antibodies and fragments thereof, include in vitro and in vivo applications well known and standard to the skilled artisan and based on the present description. Diagnostic assays and kits for in vitro assessment and evaluation of tumor and cancer status, may be utilized to diagnose, evaluate and monitor patient samples including those known to have or suspected of having cancer, a precancerous condition, a condition related to hyperproliferative cell growth or from a tumor sample. The assessment and evaluation of cancer, tumor and metastatic disease status is also useful in determining the suitability of a patient for a clinical trial of a drug or for the administration of a particular chemotherapeutic agent or targeted inhibitor, for example a specific antibody versus a different agent or binding member or ligand. This type of diagnostic monitoring and assessment is already in practice utilizing antibodies against the HER2 protein in breast cancer (Hercep Test, Dako Corporation), where the assay is also used to evaluate patients for antibody therapy using Herceptin. In vivo applications include imaging of tumors or assessing cancer status of individuals, including radioimaging.

The antibody used in the diagnostic methods of this invention may be a monoclonal antibody, and may be a human antibody. The antibody may be a single chain chain antibody or domain antibody. In addition, the antibody molecules used herein can be in the form of Fab, Fab′, F(ab′)₂ or F(v) portions of whole antibody molecules, particularly Fab. As described in detail above, antibody(ies) to phosphorylated PTEN<particularly Y240 phosphorylated PTEN, can be produced and isolated by standard methods including the phage display techniques and mutagenesis and recombinant techniques.

The presence of phosphorylated PTEN, particularly Y240 phosphorylated PTEN, in cells can be ascertained by the usual in vitro or in vivo immunological procedures applicable to such determinations. A number of useful procedures are known. The procedures and their application are all familiar to those skilled in the art and accordingly may be utilized within the scope of the present invention. The “competitive” procedure is described in U.S. Pat. Nos. 3,654,090 and 3,850,752. The “sandwich” procedure, is described in U.S. Pat. Nos. RE 31,006 and 4,016,043. Still other procedures are known such as the “double antibody,” or “DASP” procedure.

In a further embodiment of this invention, commercial test kits suitable for use by a medical specialist may be prepared to determine the presence or absence of aberrant expression of including but not limited to amplified and/or an mutation, in suspected target cells. In accordance with the testing techniques discussed above, one class of such kits will contain at least the labeled or its binding partner, for instance an antibody specific thereto, and directions, of course, depending upon the method selected, e.g., “competitive,” “sandwich,” “DASP” and the like. The kits may also contain peripheral reagents such as buffers, stabilizers, etc.

Accordingly, a test kit may be prepared for the demonstration of the presence of or elevated levels of phosphorylated PTEN, particularly Y240 phosphorylated PTEN, comprising:

(a) a predetermined amount of at least one labeled immunochemically reactive component obtained by the direct or indirect attachment of a PTEN antibody, particularly a Y240 PTEN specific antibody, to a detectable label;

(b) other reagents; and

(c) directions for use of said kit.

In an aspect of the invention, the antibody or fragment of the invention is reactive with or capable of binding phosphorylated PTEN, particularly Y240 phosphorylated PTEN. In a further aspect the antibody or fragment does not react with, does not bind to PTEN wherein Y240 is not phosphorylated. Thus, the antibody specifically recognizes PTEN post-translationally modified at Y240 by phosphorylation of that tyrosine. The antibody may be used in Western blots, immunoblotting, immunohistochemistry, immunofluorescense, FACS sorting, cell binding, cell marker studies to identify, characterize, quantitate and/or isolate phosphorylated. PTEN, particularly Y240 phosphorylated PTEN. The antibody may be utilized to isolate and/or mark or pinpoint cells or tumors which are expressing or have Y240 phosphorylated PTEN.

Panels of monoclonal antibodies recognizing PTEN, including or specifically human PTEN, can be screened for various properties; i.e., phosphospecificity, isotype, epitope, affinity, etc. Of particular interest are antibodies that react with phosphorylated PTEN and do not react with PTEN which is not phosphorylated at Y240. Of relevance are antibodies which react with or recognize phosphorylated PTEN having altered activity, e.g. which upon phosphorylated is rendered inactive or less active as a tumor suppressor, or results in reduced sensitivity to or resistance to cancer mediators, such as EGFR inhibitors. Such antibodies can be readily identified and/or screened in assays, including utilizing wild type PTEN and mutant PTEN whish cannot be phosphorylated (Y240F for instance) as described herein, including in combination with src-family kinase(s).

The PTEN antibody or phosphospecific PTEN antibody may be utilized in diagnostic and therapeutic monitoring of PTEN post-translational state(s) and/or of PTEN expression, alone or in combination with the assessment of other cancer markers or in monitoring expression of other polypeptides or enzymes. For example, but not by way of limitation, PTEN expression and phosphorylation state may be assessed using one or more other phosphorylation state-specific antibody (PSSA), including any of various cancer-associated or cancer-relevant phosphoproteins. PPSA antibodies are available against various other cellular targets and proteins/enzymes, including those relevant to cancer, and particularly in view of the role of protein phosphorylation in cancer cell growth and survival signaling (reviewed in Mandell, J W (2003) Am J Pathol 163(5):1687-1698). PPSA antibodies evaluated for correlation with cancer-related phoenotypes include those directed against ErbB-2, EGFR, ERK/MAP kinase, AKT, STAT3/6, P53, Rb, and c-jun.

For example, Marks et al (Bio/Technology, 1992, 10:779-783) describe methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR/CDRs. Marks et al further describe how this repertoire may be combined with a CDR of a particular antibody. The repertoire may then be displayed in a suitable host system such as the phage display system of WO92/01047 so that suitable specific binding members may be selected. A repertoire may consist of from anything from 10⁴ individual members upwards, for example from 10⁶ to 10⁸ or 10¹⁰ members. Analogous shuffling or combinatorial techniques are also disclosed by Stemmer (Nature, 1994, 370:389-391), who describes the technique in relation to a β-lactamase gene but observes that the approach may be used for the generation of antibodies.

A further alternative is to generate novel VH or VL regions carrying CDR-derived sequences of antibodies of the invention using random mutagenesis of, for example, the Ab VH or VL genes to generate mutations within the entire variable domain. Such a technique is described by Gram et al (1992, Proc. Natl. Acad. Sci., USA, 89:3576-3580), who used error-prone PCR. Another method which may be used is to direct mutagenesis to CDR regions of VH or VL genes. Such techniques are disclosed by Barbas et al, (1994, Proc. Natl. Acad. Sci., USA, 91:3809-3813) and Schier et al (1996, J. Mol. Biol. 263:551-567).

All the above described techniques are known as such in the art and in themselves do not form part of the present invention. The skilled person will be able to use such techniques to provide specific binding members of the invention using routine methodology in the art.

A substantial portion of an immunoglobulin variable domain will comprise at least the three CDR regions, together with their intervening framework regions. Preferably, the portion will also include at least about 50% of either or both of the first and fourth framework regions, the 50% being the C-terminal 50% of the first framework region and the N-terminal 50% of the fourth framework region. Additional residues at the N-terminal or C-terminal end of the substantial part of the variable domain may be those not normally associated with naturally occurring variable domain regions. For example, construction of specific binding members of the present invention made by recombinant DNA techniques may result in the introduction of N- or C-terminal residues encoded by linkers introduced to facilitate cloning or other manipulation steps. Other manipulation steps include the introduction of linkers to join variable domains of the invention to further protein sequences including immunoglobulin heavy chains, other variable domains (for example in the production of diabodies) or protein labels as provided herein and/or known to those of skill in the art.

Phage display screening methods may be employed using the so-called hierarchical dual combinatorial approach as disclosed in U.S. Pat. No. 5,969,108 in which an individual colony containing either an H or L chain clone is used to infect a complete library of clones encoding the other chain (L or H) and the resulting two-chain specific binding member is selected in accordance with phage display techniques such as those described in that reference. This technique is also disclosed in Marks et al, ibid Phage library and phage display selection systems and techniques are also provided herein.

The antibodies, or any fragments thereof, may be conjugated or recombinantly fused to any cellular toxin, bacterial or other, e.g. pseudomonas exotoxin, ricin, or diphtheria toxin. The part of the toxin used can be the whole toxin, or any particular domain of the toxin. Such antibody-toxin molecules have successfully been used for targeting and therapy of different kinds of cancers, see e.g. Pastan, Biochim Biophys Acta. 1997 Oct. 24; 1333(2):C1-6; Kreitman et al., N Engl J Med. 2001 Jul. 26; 345(4):241-7; Schnell et al., Leukemia. 2000 January; 14(1):129-35; Ghetie et al., Mol Biotechnol. 2001 July; 18(3):251-68.

Bi- and tri-specific multimers can be formed by association of different scFv molecules and have been designed as cross-linking reagents for T-cell recruitment into tumors (immunotherapy), viral retargeting (gene therapy) and as red blood cell agglutination reagents (immunodiagnostics), see e.g. Todorovska et al., J Immunol Methods. 2001 Feb. 1; 248(1-2):47-66; Tomlinson et al., Methods Enzymol. 2000; 326:461-79; McCall et al., J Immunol. 2001 May 15; 166(10):6112-7.

Fully human antibodies can be prepared by immunizing transgenic mice carrying large portions of the human immunoglobulin heavy and light chains. These mice, examples of such mice are the Xenomouse™ (Abgenix, Inc.) (U.S. Pat. Nos. 6,075,181 and 6,150,584), the HuMAb-Mouse™ (Medarex, Inc./GenPharm) (U.S. Pat. Nos. 5,545,806 and 5,569,825), the TransChromo Mouse™ (Kirin) and the KM Mouse™ (Medarex/Kirin), are well known within the art. Antibodies can then be prepared by, e.g. standard hybridoma technique or by phage display. These antibodies will then contain only fully human amino acid sequences. Fully human antibodies can also be generated using phage display from human libraries. Phage display may be performed using methods well known to the skilled artisan, and as provided herein as in Hoogenboom et al and Marks et al (Hoogenboom H R and Winter G. (1992) J Mol Biol. 227(2):381-8; Marks J D et al (1991) J Mol Biol. 222(3):581-97; and also U.S. Pat. Nos. 5,885,793 and 5,969,108).

Antibodies of the invention may be labelled with a detectable or functional label. Detectable labels include, but are not limited to, radiolabels such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc and ¹⁸⁶Re, which may be attached to antibodies of the invention using conventional chemistry known in the art of antibody imaging. Labels also include fluorescent labels (for example fluorescein, rhodamine, Texas Red) and labels used conventionally in the art for MRI-CT imaging. They also include enzyme labels such as horseradish peroxidase, β-glucoronidase, β-galactosidase, urease. Labels further include chemical moieties such as biotin which may be detected via binding to a specific cognate detectable moiety, e.g. labelled avidin. Functional labels include substances which are designed to be targeted to the site of a tumor to cause destruction of tumor tissue. Such functional labels include cytotoxic drugs such as 5-fluorouracil or ricin and enzymes such as bacterial carboxypeptidase or nitroreductase, which are capable of converting prodrugs into active drugs at the site of a tumor.

Also, antibodies including fragments thereof, and drugs that modulate the production or activity of the antibodies and/or their subunits may possess certain diagnostic applications and may for example, be utilized for the purpose of detecting and/or measuring conditions such as cancer, precancerous lesions, conditions related to or resulting from hyperproliferative cell growth or the like. For example, the specific binding members, antibodies or their subunits may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or antagonize the activity(ies) of the antibodies or peptides of the invention may be discovered or synthesized, and may be used in diagnostic and/or therapeutic protocols.

The radiolabelled phosphospecific binders, particularly antibodies and fragments thereof, are useful in in vitro diagnostics techniques and in in vivo radioimaging techniques and in radioimmunotherapy. In the instance of in vivo imaging, the specific binders of the present invention may be conjugated to an imaging agent rather than a radioisotope(s), including but not limited to a magnetic resonance image enhancing agent, wherein for instance an antibody molecule is loaded with a large number of paramagnetic ions through chelating groups. Examples of chelating groups include EDTA, porphyrins, polyamines crown ethers and polyoximes. Examples of paramagnetic ions include gadolinium, iron, manganese, rhenium, europium, lanthanium, holmium and ferbium. In a further aspect of the invention, radiolabelled specific binding members, particularly antibodies and fragments thereof, particularly radioimmunoconjugates, are useful in radioimmunotherapy, particularly as radiolabelled antibodies for cancer therapy. In a still further aspect, the radiolabelled specific binders, particularly antibodies and fragments thereof, are useful in radioimmuno-guided surgery techniques, wherein they can identify and indicate the presence and/or location of cancer cells, precancerous cells, tumor cells, and hyperproliferative cells, prior to, during or following surgery to remove such cells.

Immunoconjugates or antibody fusion proteins of the present invention, wherein the specific binders, particularly antibodies and fragments thereof, of the present invention are conjugated or attached to other molecules or agents further include, but are not limited to binding members conjugated to a chemical ablation agent, toxin, immunomodulator, cytokine, cytotoxic agent, chemotherapeutic agent or drug.

Radioimmunotherapy (RAIT) has entered the clinic and demonstrated efficacy using various antibody immunoconjugates. ¹³¹I labeled humanized anti-carcinoembryonic antigen (anti-CEA) antibody hMN-14 has been evaluated in colorectal cancer (Behr T M et al (2002) Cancer 94(4 Suppl):1373-81) and the same antibody with ⁹⁰Y label has been assessed in medullary thyroid carcinoma (Stein R et al (2002) Cancer 94(1):51-61). Radioimmunotherapy using monoclonal antibodies has also been assessed and reported for non-Hodgkin's lymphoma and pancreatic cancer (Goldenberg D M (2001) Crit Rev Oncol Hematol 39(1-2):195-201; Gold D V et al (2001) Crit Rev Oncol Hematol 39 (1-2) 147-54). Radioimmunotherapy methods with particular antibodies are also described in U.S. Pat. Nos. 6,306,393 and 6,331,175. Radioimmunoguided surgery (RIGS) has also entered the clinic and demonstrated efficacy and usefulness, including using anti-CEA antibodies and antibodies directed against tumor-associated antigens (Kim J C et al (2002) Int J Cancer 97(4):542-7; Schneebaum S et al (2001) World J Surg 25(12):1495-8; Avital S et al (2000) Cancer 89(8):1692-8; McIntosh D G et al (1997) Cancer Biother Radiopharm 12 (4):287-94).

In vivo animal models of cancer or animal xenograft studies may be utilized by the skilled artisan to further or additionally screen, assess, and/or verify the specific binders and antibodies or fragments thereof of the present invention, including further assessing PTEN phosphorylation in vivo and predicting prognosis or therapeutic and drug sensitivity. Such animal models include, but are not limited to models of cancer, including EGFR-mediated cancers, glioblastoma, breast cancer, lung cancer, etc. Models of cancers whose progression, migration and/or invasion involves, is facilitated by, or is associated with PTEN suppression, EGFR, PI3K/AKT, Src, are particularly susceptible to and targeted by the antibodies of the present invention. Such cancers include epithelial cancers, including breast, lung, colorectal and ovarian cancer, melanoma, head and neck cancer.

Antibodies of the present invention may be administered to a patient in need of treatment via any suitable route, including by injection intramuscularly, into the bloodstream or CSF, or directly into the site of the tumor. The precise dose will depend upon a number of factors, including whether the antibody is for diagnosis or for treatment, the size and location of the tumor, the precise nature of the antibody (whether whole antibody, fragment, diabody, etc), and the nature of the detectable or functional label attached to the antibody. Where a radionuclide is used for therapy, a suitable maximum single dose may be about 45 mCi/m², to a maximum of about 250 mCi/m². Preferable dosage is in the range of 15 to 40 mCi, with a further preferred dosage range of 20 to 30 mCi, or 10 to 30 mCi. Such therapy may require bone marrow or stem cell replacement. A typical antibody dose for either tumor imaging or tumor treatment will be in the range of from 0.5 to 40 mg, preferably from 1 to 4 mg of antibody in F(ab′)₂ form. Naked antibodies are preferably administered in doses of 20 to 1000 mg protein per dose, or 20 to 500 mg protein per dose, or 20 to 100 mg protein per dose. This is a dose for a single treatment of an adult patient, which may be proportionally adjusted for children and infants, and also adjusted for other antibody formats, in proportion for example to molecular weight. Treatments may be repeated at daily, twice-weekly, weekly or monthly intervals, at the discretion of the physician.

Antibodies and fragments thereof of the present invention will usually be administered in the form of a pharmaceutical composition or pharmaceutically-acceptable compositions, which may comprise at least one component in addition to the antibody. Thus pharmaceutical compositions according to the present invention, and for use in accordance with the present invention, may comprise, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous, or by deposition at a tumor site.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. For intravenous, injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

A composition may be administered alone or in combination with other treatments, therapeutics or agents, either simultaneously or sequentially dependent upon the condition to be treated. In addition, the present invention contemplates and includes compositions comprising the binding member, particularly antibody or fragment thereof, herein described and other agents or therapeutics such as anti-cancer agents or therapeutics, hormones, anti-mitotic agents, anti-apoptotic agents, antibodies, or immune modulators. More generally these anti-cancer agents may be but are not limited to tyrosine kinase inhibitors or phosphorylation cascade inhibitors, post-translational modulators, cell growth or division inhibitors (e.g. anti-mitotics), or signal transduction inhibitors. Other treatments or therapeutics may include the administration of suitable doses of pain relief drugs such as non-steroidal anti-inflammatory drugs (e.g. aspirin, paracetamol, ibuprofen or ketoprofen) or opiates such as morphine, or anti-emetics. The composition can be administered in combination (either sequentially (i.e. before or after) or simultaneously) with tyrosine kinase inhibitors (including, but not limited to AG1478 and ZD1839, STI571, OSI-774, SU-6668), doxorubicin, temozolomide, cisplatin, carboplatin, nitrosoureas, procarbazine, vincristine, hydroxyurea, 5-fluoruracil, cytosine arabinoside, cyclophosphamide, epipodophyllotoxin, carmustine, lomustine, and/or other chemotherapeutic agents. Thus, these agents may be specific anti-cancer agents, or immune cell response modulators or may be more general anti-cancer and anti-neoplastic agents such as doxorubicin, cisplatin, temozolomide, nitrosoureas, procarbazine, vincristine, hydroxyurea, 5-fluoruracil, cytosine arabinoside, cyclophosphamide, epipodophyllotoxin, carmustine, or lomustine. In addition, the composition may be administered with hormones such as dexamethasone, immune modulators, such as interleukins, tumor necrosis factor (TNF) or other growth factors, colony stimulating factors, or cytokines which stimulate the immune response and reduction or elimination of cancer cells or tumors. The composition may also be administered with, or may include combinations along with other anti-tumor antigen antibodies.

In addition, the present invention contemplates and includes therapeutic compositions for the use of the antibodies in combination with conventional radiotherapy.

The preparation of therapeutic compositions which contain polypeptides, analogs or active fragments as active ingredients is well understood in the art. Typically, such compositions are prepared as injectables, either as liquid solutions or suspensions. However, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic antibody- or active fragment-containing compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated or tumor antigen binding capacity desired. Precise amounts of active ingredient required to be administered depend on the judgment of the practitioner and are peculiar to each individual. Suitable regimes for initial administration and follow on administration are also variable, and may include an initial administration followed by repeated doses at one or more hour intervals by a subsequent injection or other administration. Alternatively, continuous intravenous infusion sufficient to maintain appropriate and sufficient concentrations in the blood or at the site of desired therapy are contemplated.

In accordance with the above, an assay system for screening potential drugs effective to modulate the phosphorylation or activity of PTEN and/or the activity or binding of the antibody of the present invention may be prepared. The antigen peptide or the binding member or antibody may be introduced into a test system, and the prospective drug may also be introduced into the resulting cell culture, and the culture thereafter examined to observe any changes in the activity of the cells, binding of the antibody, or amount and extent of phosphorylated PTEN due either to the addition of the prospective drug alone, or due to the effect of added quantities of the known agent(s).

The present invention also provides constructs in the form of plasmids, vectors, transcription or expression cassettes which comprise at least one polynucleotide. The present invention also provides a recombinant host cell which comprises one or more constructs as above. A nucleic acid encoding any specific binding member or antibody as provided itself forms an aspect of the present invention, as does a method of production of the specific binding member which method comprises expression from encoding nucleic acid therefor. Expression may conveniently be achieved by culturing under appropriate conditions recombinant host cells containing the nucleic acid. Following production by expression a specific binding member may be isolated and/or purified using any suitable technique, then used as appropriate.

Specific binding members and encoding nucleic acid molecules and vectors according to the present invention may be provided isolated and/or purified, e.g. from their natural environment, in substantially pure or homogeneous form, or, in the case of nucleic acid, free or substantially free of nucleic acid or genes origin other than the sequence encoding a polypeptide with the required function. Nucleic acid according to the present invention may comprise DNA or RNA and may be wholly or partially synthetic.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, cancer cells, ovarian cancer cells and many others. A common, preferred bacterial host is E. coli. The expression of antibodies and antibody fragments in prokaryotic cells such as E. coli is well established in the art. Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology, Second Edition, Ausubel et al. eds., John Wiley & Sons, 1992. The disclosures of Sambrook et al. and Ausubel et al. are incorporated herein by reference.

Thus, a further aspect of the present invention provides a host cell containing nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene. The present invention also provides a method which comprises using a construct as stated above in an expression system in order to express a specific binding member or polypeptide as above.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host. A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMB9 and their derivatives, plasmids such as RP4; phage DNAs, e.g., the numerous derivatives of phage λ, e.g., NM989, and other phage DNA, e.g., M13 and filamentous single stranded phage DNA; yeast plasmids such as the 2u plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, YB/20, NSO, SP2/0, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

Glioblastoma multiforme (GBM) is the most aggressive of the astrocytic malignancies and the most common intracranial tumour in adults. Those patients with GBMs expressing the mutant epidermal growth factor receptor, EGFRvIII, together with the PTEN tumor suppressor show initial clinical responses to the EGFR kinase inhibitors erlotinib or gefitinib^{2, 3}. However, not all patients whose tumors have this genotype responded and, for those who did, the responses were often short-lived before tumors acquired resistance. Using mass spectrometry, we have mapped Src-induced phosphorylation sites on PTEN. For the first time, we directly demonstrate tyrosine phosphorylation of PTEN in human tumors, particularly in those GBMs that retain wild type PTEN but fail to respond to EGFR inhibitors. Using a PTEN allele in which a single tyrosine residue, Y240 is mutated to phenylalanine, we show that the modification at this site modulates EGFRvIII signaling and the cellular response to Erlotinib. Thus, in addition to loss or mutation of the PTEN gene^{2, 4}, resistance to targeted therapy can also be driven by Src-mediated PTEN phosphorylation. These findings suggest that blocking PTEN phosphorylation by suppression of Src activity may re-sensitize PTEN-intact tumors to EGFR inhibitors, a class of drugs which hold promise for the treatment of a wide range of cancers⁵.

The PTEN tumor suppressor gene is lost or mutated in approximately 40% of GBMs, leading to aberrant activation of highly oncogenic PI3K/Akt signaling and other downstream pathways^{1, 6}. In addition to this regulatory function, it is becoming increasingly clear that, even in cells harboring the wild type PTEN gene, its protein function can be affected by a number of posttranslational modifications, including phosphorylation, acetylation and ubiquitination⁷. This complicates the significance of detecting wild type PTEN in tumor cells, as it is not necessarily an indication that PTEN function has been retained. This lends increasing importance to being able to detect modified PTEN in human tumors and to discover the molecules that, as PTEN-modifiers, may represent useful therapeutic targets to restore PTEN tumor suppressor function.

Phosphorylation of PTEN by src-family kinases has been proposed to inhibit its function in a number of ways, including loss of membrane interaction and decreased protein stability^{8, 9}, but it is not clear how this is accomplished and whether these modifications have any import for the behavior of human tumors. To investigate this, we used mass spectrometry to accurately identify tyrosine residues in PTEN that were phosphorylated following co-expression with c-src in 293T cells or incubation of recombinant purified PTEN with c-src in vitro (TABLE 1 and data not shown).

PTEN phosphorylation sites were identified by mass spectrometry. HA-FLAG-PTEN was immunoprecipitated from 293T cells expressing c-src using anti-FLAG-M2-agarose (Sigma). Following elution from the antibody, PTEN was digested and phospho-peptides were purified by IMAC before mass spectrometry (see methods below). The results are shown in Table 1 below. The peptides below correspond to SEQ ID NOS: 2-12.

TABLE 1
Identification of PTEN Phosphorylation Sites by Mass Spectrometry

| Probability | Xcorr | Deltacn | Sprank | Ions | Peptide | Site |
|---|---|---|---|---|---|---|
| 0.8072 | 2.417 | 0.11 | 5 | 23/52 | R.LEGVY[243.03]RNNIDDVVR.F | Y46 |
| 0.9995 | 3.07 | 0.323 | 1 | 13/14 | K.IY[243.03]NLC[160.03]AER.H | Y68 |
| 0.9986 | 2.671 | 0.271 | 1 | 13/14 | K.IY[243.03]NLC[160.03]AER.H | Y68 |
| 0.8688 | 3.439 | 0.34 | 1 | 26/44 | K.NHYKIY[243.03]NLC[160.03]AER.H | Y68 |
| 1 | 3.828 | 0.373 | 1 | 18/22 | K.AQEALDFY[243.03]GEVR.T | Y155 |
| 0.9994 | 3.392 | 0.341 | 1 | 17/22 | K.AQEALDFY[243.03]GEVR.T | Y155 |
| 0.9736 | 3.063 | 0.37 | 1 | 17/18 | R.Y[243.03]VYYYSYLLK.N | Y174 |
| 0.9983 | 3.059 | 0.179 | 1 | 27/64 | K.FMY[243.03]FEFPQPLPVC[160.03]GDIK.V | Y240 |
| 0.9999 | 4.228 | 0.252 | 2 | 25/52 | R.ADNDKEY[243.03]LVLTLTK.N | Y315 |
| 0.9235 | 3.983 | 1 | 1 | 38/112 | K.TVEEPSNPEASSSTSVT[181.01]PDVS[167.00]DNEPDHYR.Y | T366, |
| | | | | | | S370 |
| 0.8171 | 3.472 | 0.213 | 3 | 24/92 | R.YSDTTDS[167.00]DPENEPFDEDQHTQITK.V | S385 |

We identified four sites of tyrosine phosphorylation in the PTEN phosphatase domain (Y46, Y68, Y155 and Y174) and two sites in the PTEN C2 domain (Y240 and Y315). Missense mutations at both Y68 and Y155 have been detected in human GBMs and in the germlines of patients with genetic disorders caused by a loss of PTEN function, such as Cowden Syndrome¹⁰, suggesting that src may directly inhibit PTEN phosphatase activity by targeting these sites. Our detection of phosphorylation on tyrosines 240 and 315 is also consistent with an earlier study in which mutation of these sites led to a reduction in overall PTEN tyrosine phosphorylation upon co-expression with the related src-family kinase, Lck¹¹.

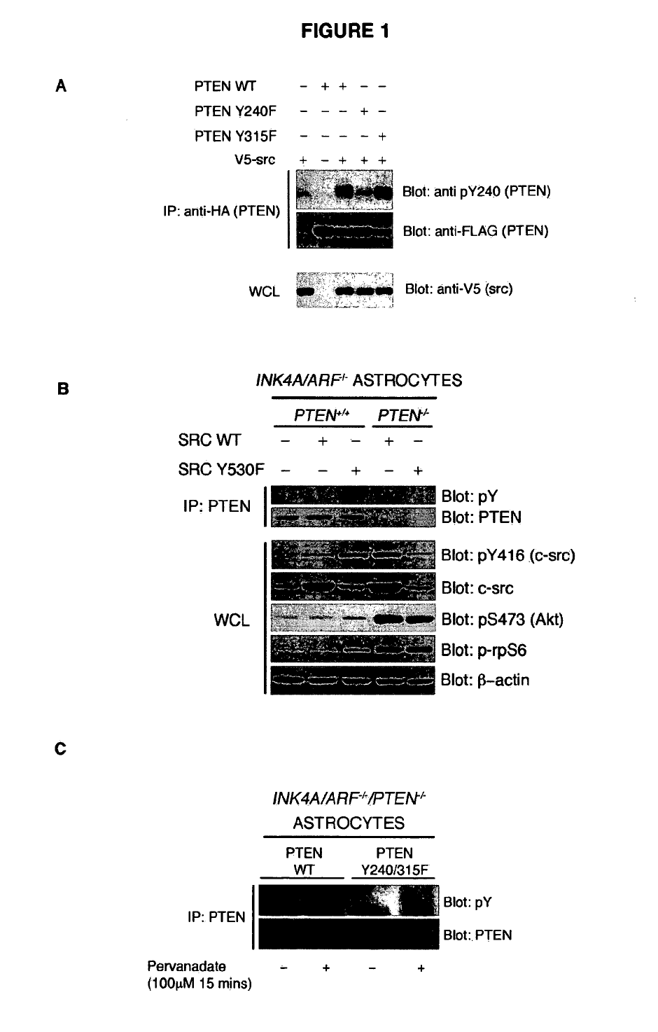

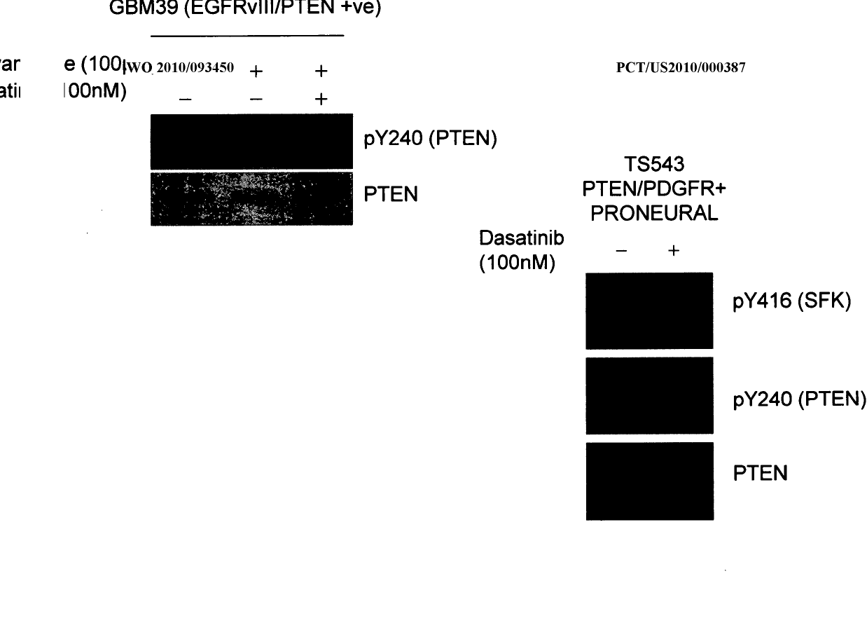

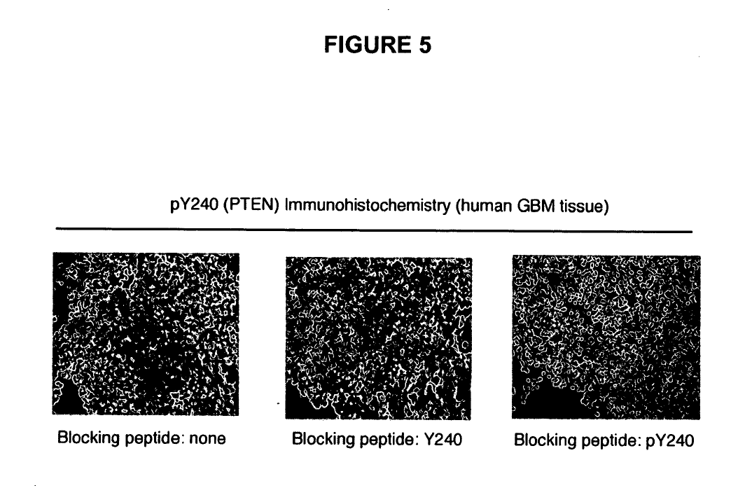

In order to study the phosphorylation of endogenous PTEN in cells and tissues we attempted to produce phospho-specific antibodies against these sites, and were successful in generating antibodies against phosphorylated tyrosine 240 (pY240). The specificity of these antibodies was demonstrated by western blotting following co-expression of c-src with PTEN bearing single amino acid substitutions at tyrosine phosphorylation sites Y240F or Y315F (FIG. 1A). Moreover, peptide competition experiments employing immunohistochemistry of tissue sections from glioblastomas with confirmed wild-type PTEN expression, showed that only the phosphorylated peptide could compete away the immunoreactivity (FIG. 5).

We next assessed whether src can promote tyrosine phosphorylation of endogenous PTEN in astrocytes, a candidate cell type of origin for GBM¹². Astrocytes from ink4a/arf^{-/-} mice carrying ‘foxed’ PTEN alleles (PTEN^{loxP/loxP}) (from which the PTEN alleles had been excised by CRE-recombinase where indicated PTEN^{-/-})¹³ were infected with retroviruses encoding either wild type c-src, or a constitutively active c-src construct (Y530F) in which the C-terminal inhibitory tyrosine phosphorylation site was mutated to phenylalanine. Following serum starvation, tyrosine phosphorylation of endogenous PTEN was detected specifically in astrocytes expressing active c-src (FIG. 1B). Additionally, in agreement with the findings of Dey and co-workers¹⁴, we found evidence that PTEN may itself act as a negative regulator of c-src, as revealed by the increased Y416 phosphorylation of wild type c-src in PTEN^{-/-} astrocytes (FIG. 1B, compare lanes 2 and 4). Next, we reconstituted ink4a/arf/pten^{-/-} astrocytes expressing active c-src with either wild type or Y240/315F mutant PTEN. Tyrosine phosphorylation was detected only on the wild type protein (FIG. 1C), indicating that the PTEN C2 domain sites can indeed be targeted by src in glial cells.

Analysis of glioma cells (GBM39) in which expression of EGFRvIII has been retained by maintenance of the cells as xenografts in the flanks of nude mice¹⁵ showed that PTEN is phosphorylated on Y240 in these cells (data not shown). Treatment with the src inhibitor Dasatinib decreased PTEN Y240 phosphorylation and also decreased the phosphorylation of downstream pathway components, PRAS40 and the 40S ribosomal S6 protein to a similar extent. The dasatinib sensitivity displayed by Y240 phosphorylation in glioma cell lines was evaluated (FIG. 17). In cell line GBM39, a substantial increase in Y240 phosphorylation can be induced by the addition of pervanadate, which inhibits tyrosine phosphatases. This increase is sensitive to dasatinib. We also observed dasatinib-sensitive Y240 phosphorylation in TS543, a cell line that has been developed and maintained under ‘neurosphere’ conditions, which preserve the genetic characteristics of the cells seen in the original tumor. These results demonstrate that the PY240 phosphorylation is highly sensitive to dasatinib.

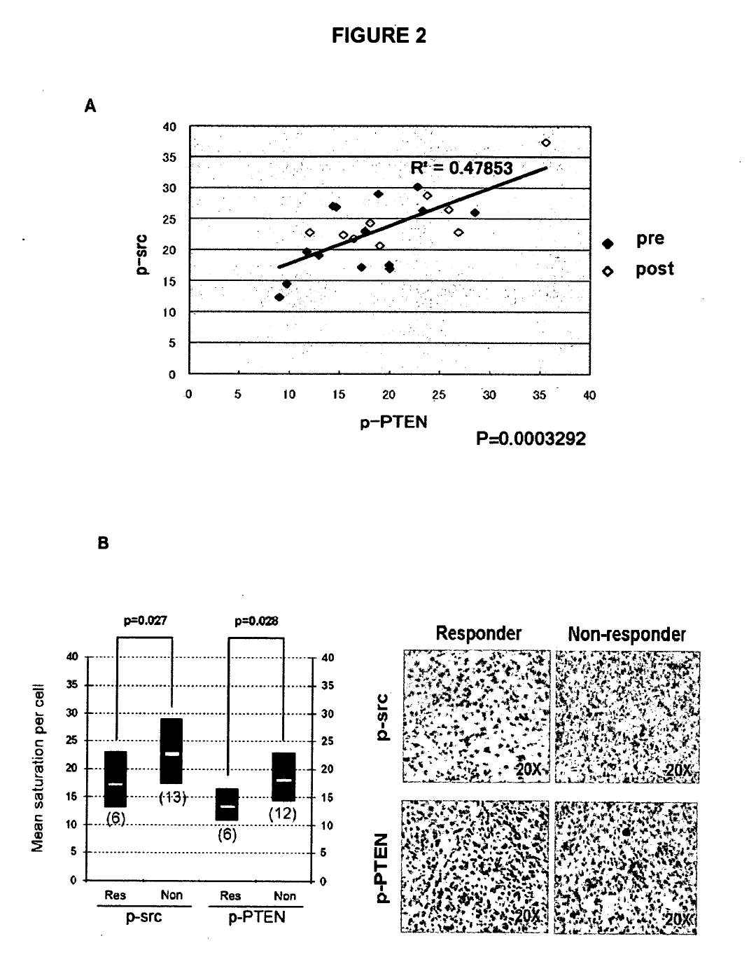

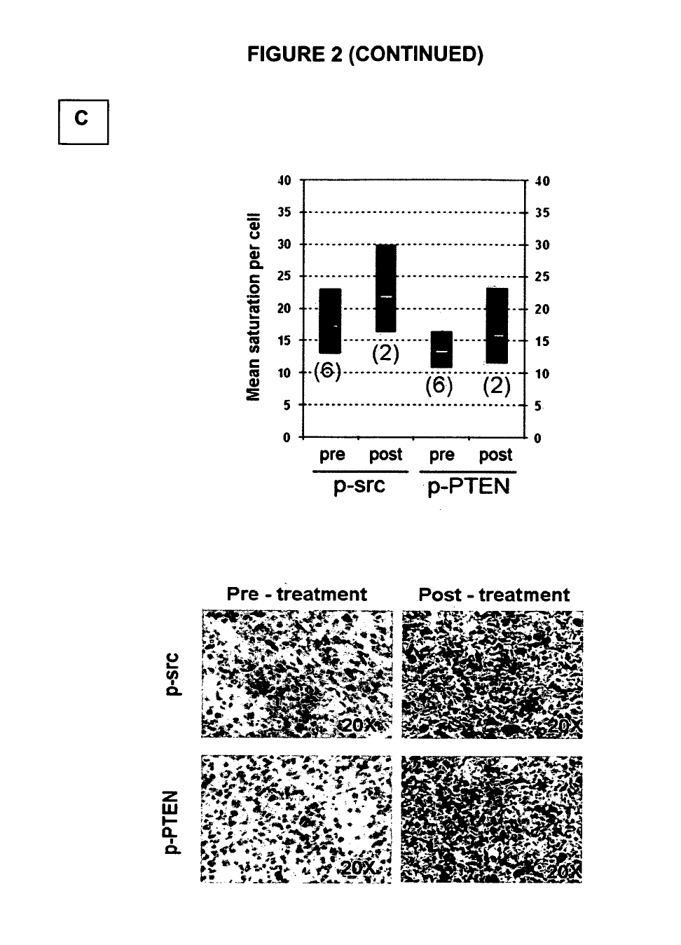

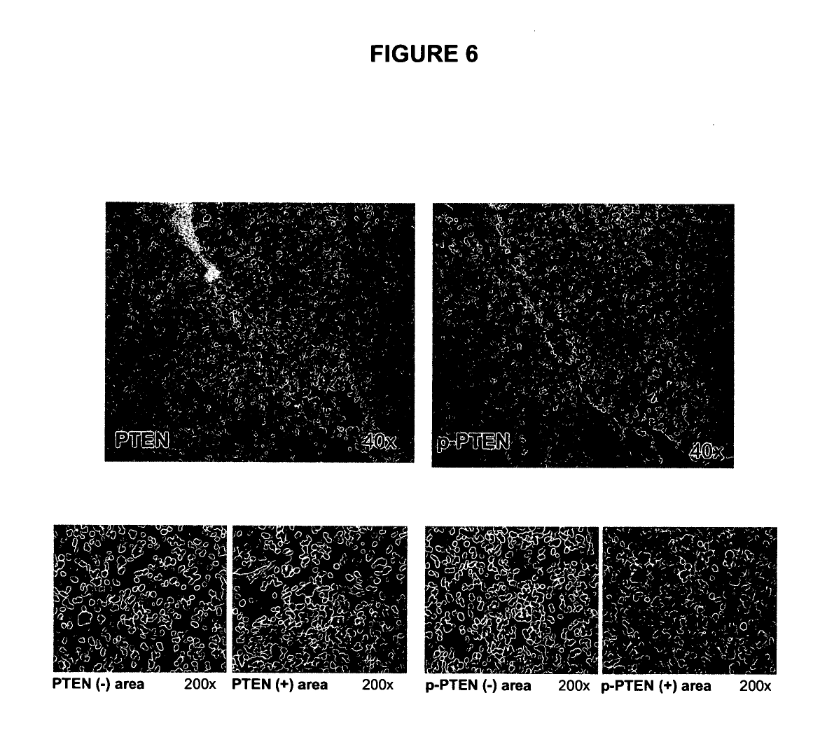

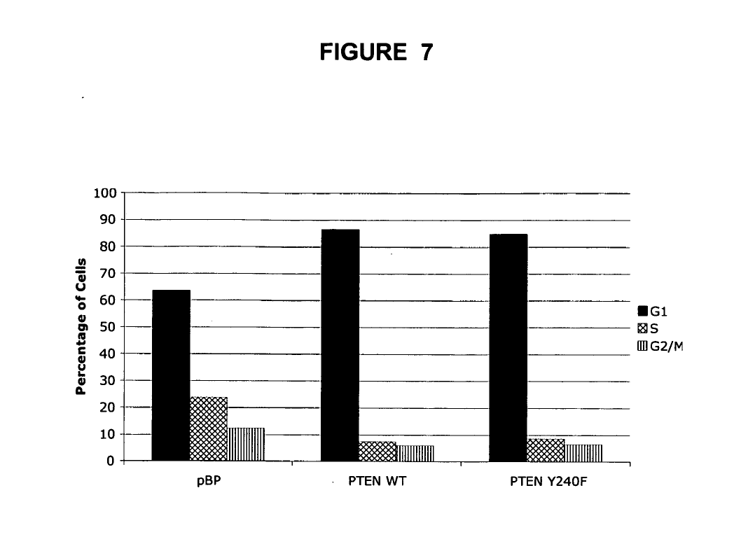

Having established the specificity of the pY240 antibodies and demonstrated phosphorylation of Y240 in glioma cell lines and astrocytes, we next analyzed PTEN phosphorylation in tumor tissue sections from GBM patients who had participated in an EGFR kinase inhibitor clinical trial². In parallel with the PTEN pY240 analysis, we also assessed the activation of src-family kinases (SFKs) in these samples using an antibody specific for the conserved catalytic domain autophosphorylation site (Y419 in human c-src). We observed a striking correlation (R²=0.47853, p<0.0004) between src family kinase activation and PTEN phosphorylation on Y240 in the patient samples (FIG. 2A). Significantly, upon comparison of the immunohistochemistry data with the patient response data from the clinical trial, we found that the tumors with high SFK phosphorylation (p=0.027) and pY240 PTEN phosphorylation (p=0.028) were those that failed to respond to EGFR inhibitors (either Erlotinib or Gefitinib), despite expressing EGFRvIII and wild type PTEN (FIG. 2B). Furthermore, we discovered that in two patients whose tumors initially responded to treatment but later acquired resistance, the SFK activity and PTEN pY240 levels were initially low but in the resistant tumors had increased to levels similar to those seen in the tumors that displayed ‘upfront resistance’⁴ (FIG. 2D). Finally, we observed strong pY240 staining in regions of residual PTEN expression in GBMs that were otherwise PTEN-null (FIG. 6).

This finding underlines the heterogeneity of GBM and suggests that PTEN function may be compromised by multiple genetic and posttranslational mechanisms within a single tumor. Although there are only a relatively small number of patients available from these trials, when taken together these findings strongly suggest a link between PTEN phosphorylation and both upfront and acquired resistance to EGFR inhibitors in GBM. Furthermore, the activation of src family kinases observed in resistant tumors is consistent with a role for alternative receptor tyrosine kinases in driving resistance, by maintaining mitogenic signaling in the presence of EGFR inhibition¹⁶.

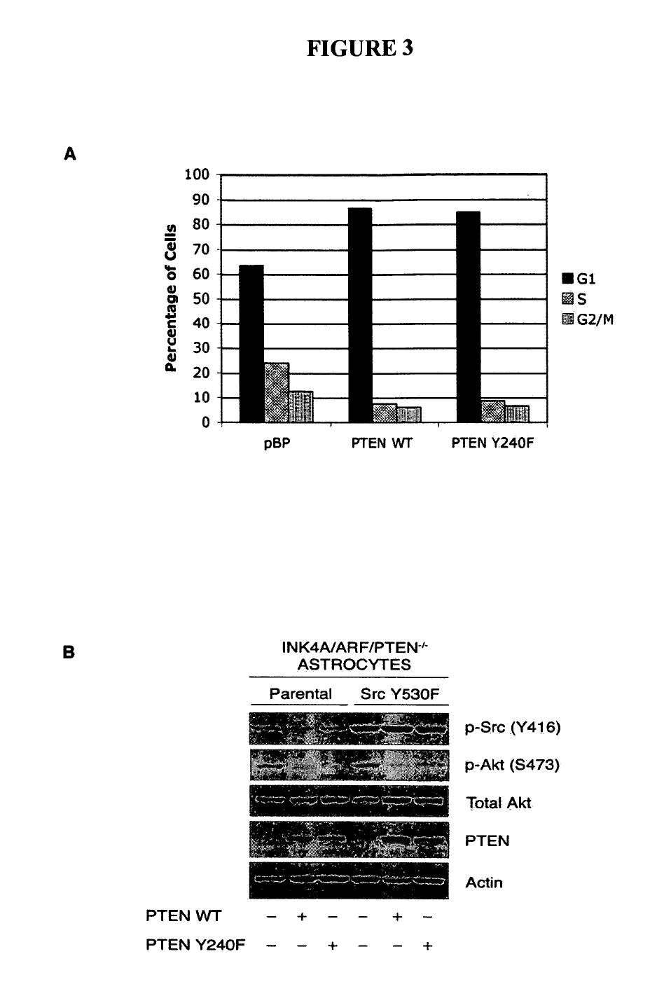

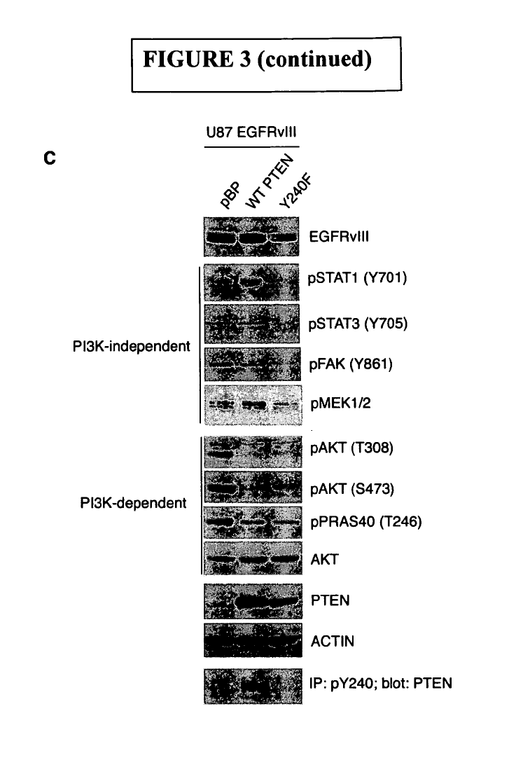

Pharmacological or genetic suppression of src family kinase activity is known to have pleiotropic effects on glioma cells^{17, 18}. Therefore, we sought to determine the specific role that PTEN phosphorylation on tyrosine 240 might play in resistance to EGFR inhibitors by introducing the non-phosphorylatable Y240F PTEN mutant allele into two relevant and different cell lines with PTEN-null backgrounds. First, we reconstituted the U87MG human glioma cell line (which harbors mutant Pten) with either wild-type PTEN or the Y240F PTEN mutant. Both WT and Y240F PTEN caused G1 arrest when expressed in U87MG cells (FIG. 3A), a well-established readout for PTEN function¹⁹. Consistent with this, both alleles displayed similar effects on the activation of AKT and downstream pathways (data not shown). In addition to U87MG cells, we also reconstituted PTEN-null mouse astrocytes with WT or Y240F PTEN and again observed a similar ability of both alleles to suppress AKT pathway activation, even in cells expressing the active src allele (FIG. 3B). The ability of PTEN to suppress AKT activation and the growth of U87MG cells expressing EGFRvIII was also assessed and found to be similar for both wild-type and Y240F alleles (FIG. 3C). Interestingly, the Y240F mutant was previously shown to be unable to suppress anchorage-independent growth or tumorigenicity of U251 cells¹¹, a line in which PTEN acts within the nucleus to effect G1 arrest independently of AKT regulation, in contrast to the requirement for its cytoplasmic localization in U87MG cells^{20, 21}. These cell type-specific effects suggest that phosphorylation of PTEN on Y240 may influence its localization or interaction with specific binding partners.

During analyses of U87MG-EGFRvIII cells reconstituted with PTEN, we found that introduction of the Y240F PTEN mutant consistently caused a reduction in the activation of PI3K-independent targets of EGFRvIII signaling including STAT1, STAT3, MEK and FAK, associated with a decrease in levels of the receptor (FIG. 3C). The involvement of PTEN Y240 phosphorylation in STAT3 regulation suggested by our data is particularly interesting given the recent report that inhibition of STAT3 sensitizes GBM cells to gefitinib²².

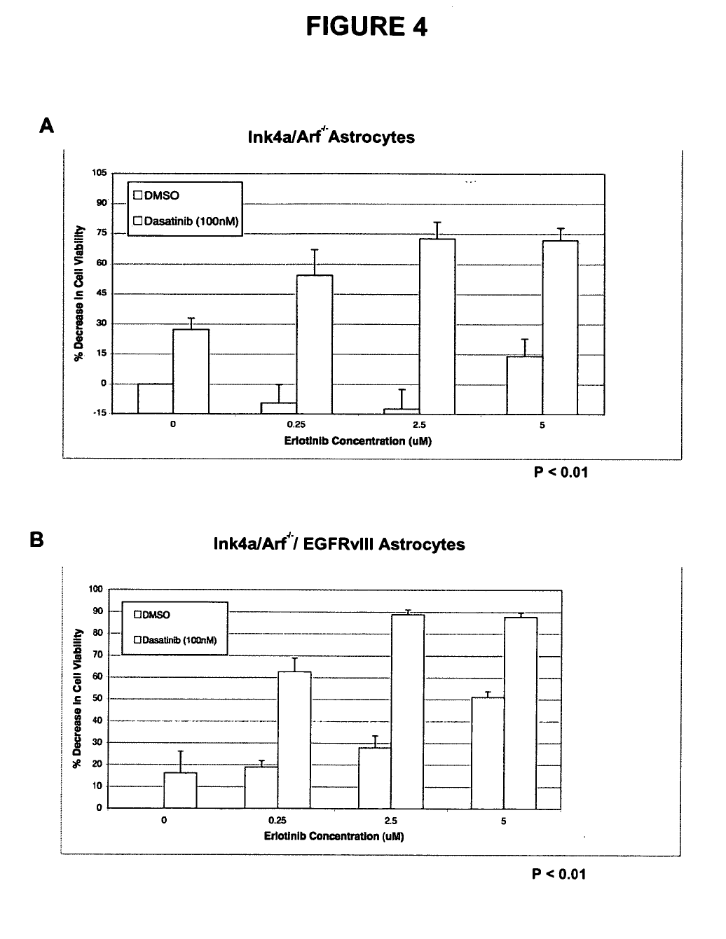

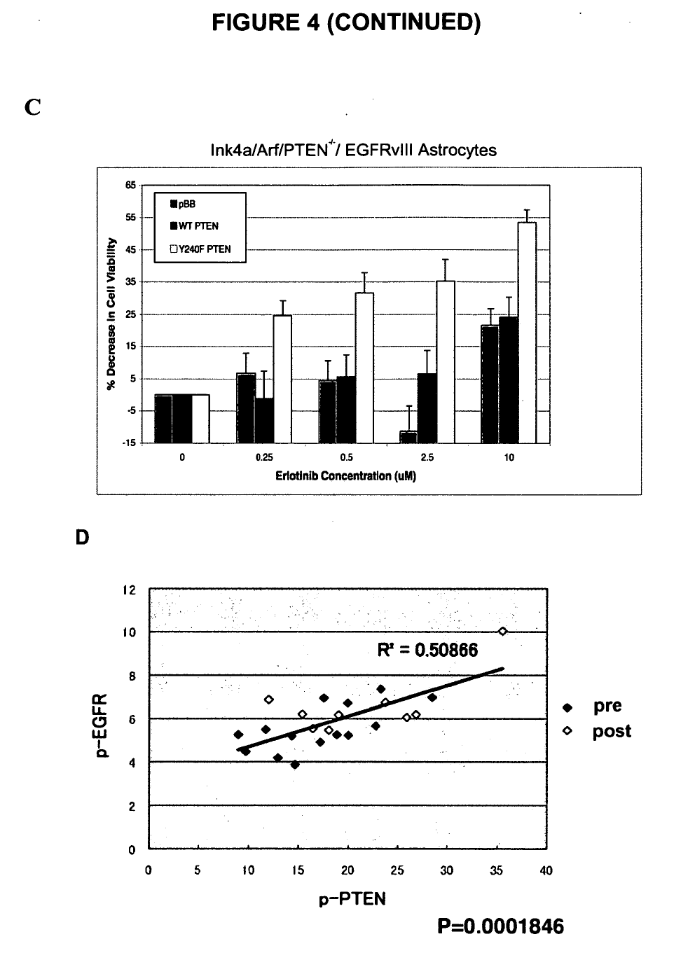

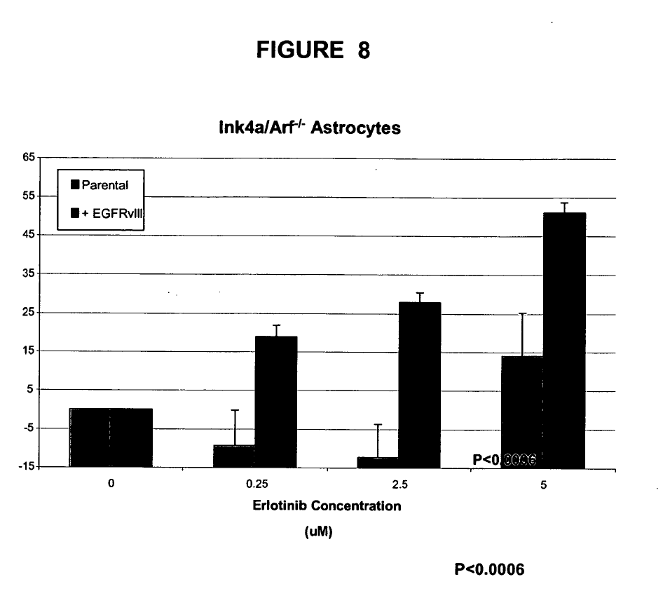

Having established that Src can phosphorylate PTEN on Y240 and that these activities occur concomitantly in human GBMs that have not responded to EGFR inhibitors, we next sought to determine whether this was due to a causal relationship. In order to test the effects of PTEN Y240 phosphorylation on sensitivity to EGFR inhibitors in a genetically defined system, we used PTEN-null astrocytes expressing EGFRvIII and reconstituted them with either WT or Y240F PTEN. We first determined that, similar to glioma cell lines² and consistent with the strong correlation in human tumors, expression of EGFRvIII significantly (p<0.0006) sensitizes astrocytes expressing WT PTEN to Erlotinib (FIG. 8). Furthermore, addition of the src/Abl inhibitor Dasatinib, sensitized both parental and EGFRvIII-expressing astrocytes to Erlotinib (FIGS. 4A and B) suggesting that SFK activity may play a role in modulating the response to EGFR inhibitors in these cells. As expected from the clinical data, PTEN-null astrocytes expressing EGFRvIII were resistant to clinically achievable doses of Erlotinib, as were EGFRvIII astrocytes reconstituted with WT PTEN (FIG. 4C). In sharp contrast, introduction of the PTEN Y240F mutant significantly sensitized these cells to sub-micromolar doses of Erlotinib (FIG. 4C). Note that levels of PTEN expression achieved by reconstitution of PTEN-null astrocytes were considerably lower than the level of PTEN found in parental astrocytes, likely accounting for the difference in Erlotinib sensitivity of these cells (data not shown).

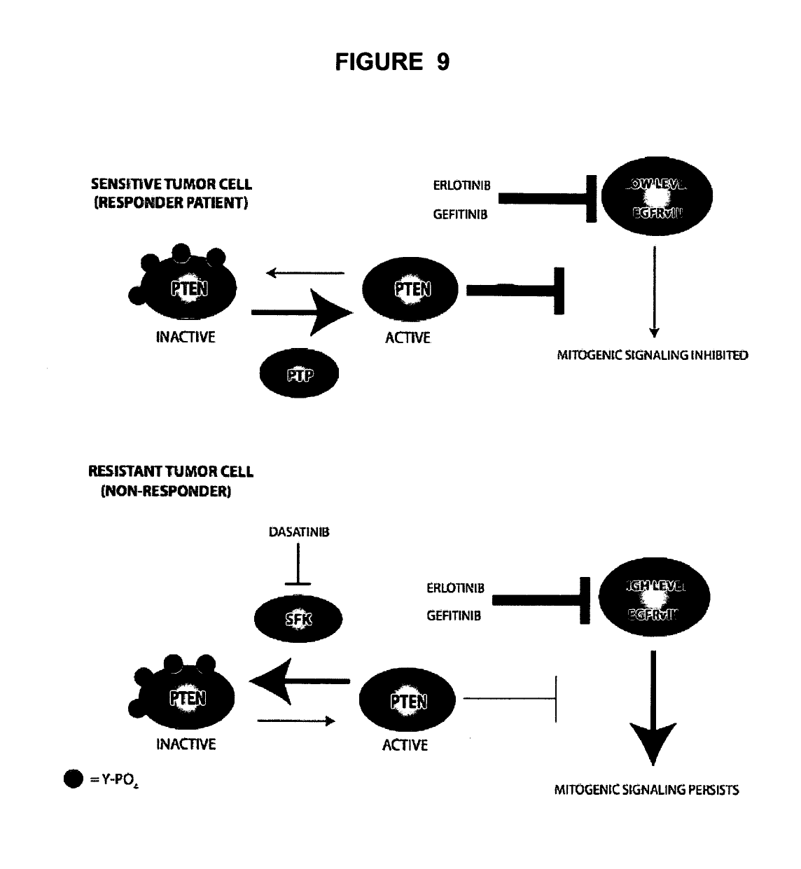

Since our data (FIG. 3C) suggested that phosphorylation of PTEN on Y240 relieves PTEN-mediated inhibition of EGFR signaling, we predicted that Y240 phosphorylation drives drug resistance through this mechanism. We therefore examined those clinical samples displaying PTEN Y240 phosphorylation and discovered a significant correlation (R²=0.50866, p<0.0002) between p-PTEN and p-EGFR, both pre- and post-treatment (FIG. 4C). Significantly, loss of PTEN in GBM by mutation or deletion is similarly shown to upregulate EGFR, through impaired turnover of the receptor. This change in receptor levels shifts the IC₅₀ for EGFR inhibition, potentially accounting for the higher activity of EGFR seen in resistant samples post-treatment (I. Mellinghoff, personal communication). Taken together with the clinical data from figure two, these data demonstrate that in addition to PTEN loss or mutation, src-dependent phosphorylation of PTEN is an important mechanism that drives resistance to EGFR inhibitors in glioblastoma patients (our model is represented in FIG. 9). Furthermore, the use of SFK inhibitors like Dasatinib may sensitize resistant tumors to EGFR inhibition, at least in part, by suppressing PTEN phosphorylation. The importance of PTEN in suppressing tumorigenesis in multiple organs suggests that these findings may apply to a wide range of cancers in which src activity is elevated in the presence of wild type PTEN, potentially suggesting new approaches for treatment.

Methods Summary

Cell Culture. U87MG cells and Ink4a/Arf^{-/-} astrocytes expressing EGFRvIII, Ink4a/Arf^{-/-}/PTEN^{loxP/loxP} astrocytes and GBM39 have all been described previously^{12, 13, 15, 23}.

Mouse Astrocytes. Ink4a/Arf^{-/-1} and Pten^{loxP/loxP 2} mice were obtained from the NCI mouse depository and intercrossed to generate described genotypes. Primary astrocytes were isolated from the neocortex of 4 day old pups, using standard astrocyte isolation culture conditions (Bachoo et al 2002). Conditional deletion of Pten^{loxP/loxP} was accomplished by infecting astrocyte cultures with adenovirus expressing Cre recombinase under the CMV promoter (Gene TransferVector Core, University of Iowa, Iowa City, Iowa). Effective Pten deletion was verified by western blot. EGFRvIII was expressed in these cells by retroviral infection as described previously (Bachoo et al 2002). (Reference: Bachoo, R. M. et al. Epidermal growth factor receptor and Ink4a/Arf: convergent mechanisms governing terminal differentiation and transformation along the neural stem cell to astrocyte axis. Cancer Cell 1, 269-77 (2002)).

GBM39 Ex Vivo Culture

Mice were sacrificed by CO₂ inhalation followed by cervical dislocation, according to institutional guidelines. Subcutaneous tumors were removed and tumor tissue was minced into 0.1-0.5 mm³ chunks in a 60 mm dish with 5 ml of freshly prepared, filter-sterilized digestion solution (12,500 units of Collagenase Type 2, 12,500 units of Collagenase Type 4, 20 mL DMEM (Serum Free), 20 uL of 1 mg/mL DNAase I). Tissue was placed into a sterile 15 ml specimen cup with an additional 10 ml of digestion solution and shaken (150 rpm) at 37 C for 3 hours before filtration through a 70/40 um filter. After centrifugation at 1000 rpm for 3 minutes, the supernatant was discarded. The pellet was resuspended in 1 ml of ACK buffer (150 mM NH₄Cl, 10 mM KHCO₃, 0.1 mM Na₂EDTA, adjusted to pH 7.2-7.4 with 1N HCl) and incubated at room temperature for 3 minutes to lyse red blood cells. Cells were pelleted by centrifugation at 1000 rpm for 3 minutes and resuspended in culture medium (DMEM containing 10% fetal bovine serum plus L-glutamine and penicillin/streptomycin). Cell viability was determined by trypan blue exclusion and cells were plated at a density of approx 10⁶ cells per 100 mm culture dish. Cells were maintained under standard cell culture conditions (37 C, 5% CO₂ in a humidified incubator) and all experiments were conducted in cells prior to p4.

Retroviral infections with pBABE-blasticidin-src, pBABE-puromycin and pBABE-blasticidin PTEN constructs were performed using the RetroMax system (Imgenex Corp.) according to the manufacturers' instructions, using the ecotropic or 10A1 envelopes for mouse astrocytes and U87MG cells respectively.

Antibodies. The polyclonal anti-pY240 site-specific antibody was raised in rabbit (Pacific Immunology, Ramona, Calif.) using the PTEN-derived phospho-peptide: TRREDKFMpYFEFPQPLPVC (SEQ ID NO: 13). Antibodies were affinity purified against both the phospho- and non-phospho peptide to eliminate reactivity against unphosphorylated PTEN. Other antibodies used were: PTEN (N-19 and A2B1) and c-src (SRC 2) (Santa Cruz); AKT, pAKT (S473), pAKT (T308), pPRAS40 (T246), p-src (Y416), pSTAT3 (Y705), pMEK1/2 (S217/221), prpS6 (S240/244) (Cell Signaling Technology); β-actin-HRP, HA (HA-7) FLAG (M2), pFAK (Y861) (Sigma); pSTAT1 (Y701) (Upstate); V5-HRP (Invitrogen); EGFR(C-13) (BD Biosciences), p-EGFR (Y1086) (Invitrogen).

Mass Spectrometry: FLAG-HA-PTEN was immunoprecipitated from 293T cells using M2-agarose (Sigma) and eluted in 5% acetic acid. IMAC purification of phospho-peptides and mass spectrometry were conducted as described²⁴.

Immunohistochemistry: Tissue sections were cut from blocks of formalin-fixed paraffin tumor tissue from glioblastoma patients treated at UCLA with the EGFR kinase inhibitors (erlotinib or gefitinib), including a subset of patients previously described². Tumor specimens were obtained according to a protocol approved by the institutional review board of UCLA. Pre-treatment tumor tissue was examined for 19 patients; post-treatment tissue was examined from 8 patients, including 7 patients for whom pre- and post-treatment tissue was available. Patients were stratified as responders (at least 25% decrease in tumor bidirectional area on MRI) or non-responders (at least 25% increase in tumor bidirectional area, as previously described². Five micron tissue sections were stained with polyclonal antibodies to phospho-Src (Tyr416, #2101, Cell Signaling Technology, Inc, USA), phospho-PTEN Y240 (see above) and phospho-EGFR (Tyr 1086, #36-9700, Invitrogen, USA). Antigen retrieval was performed using 0.01 M citrate buffer, pH 6.0 for 30 minutes in an oven. Peroxidase activity was quenched with 3% hydrogen peroxide in water. Primary antibodies were used at 1:12.5 dilution for phospho-Src and 1:25 dilution for phospho-PTEN in 10% normal goat serum in TBS-0.1% Tween and applied for 16 hours at 4° C., followed by biotinylated secondary antibodies (Vector) at 1:200 dilution for 30 minutes, and avidin-biotin complex (Elite ABC, Vector) for 40 minutes. Negative control slides received blocking serum (10% normal goat serum in TBS-0.1% Tween). Vector NovaRed was used as the enzyme substrate to visualize specific antibody localization. Slides were counterstained with Harris hematoxylin.

Image analysis-based scoring of immunohistochemistry: Digital scores for phospho-Src, phospho-PTEN, and phospho-EGFR were based on absolute staining intensity of tumor cells as quantified following false-color conversion. Phospho-Src and phospho-PTEN were scored in representative and matching areas of consecutive tissue sections using previously described algorithm². Sections were photographed using a Colorview II camera mounted on an Olympus BX41 microscope at 20× magnification. 5 images were captured per slide from representative regions of the tumor. Borders between individual cells were approximated using a separator function of the Soft Imaging Software (with the parameter of Smooth and Fine/Coarse, 2 and 10 respectively). Quantitative analysis was done using HSI color algorithm based on hue, saturation and intensity. Saturations of the separated cell in the images were quantified in the red-brown hue range to exclude the negative staining area with hematoxylin nuclear staining. To compare the staining intensity of all slides, mean saturation of total cells on each image was quantified and calculated. 2500-3000 cells per case (on average) were measured for each slide and statistical comparisons were performed using R software, using an approach previously described²⁵.

Drug sensitivity assays. Cells were plated at 1000 cells/well in 96 well plates and allowed to adhere for four hours prior to addition of Erlotinib and/or Dasatinib or solvent (DMSO) control. Cells were maintained in 5% serum during drug treatments and cell viability was measured using WST-1 assay (Clontech) following 72 hours exposure to drug.

Mouse Astrocytes. Ink4a/Arf^{-/- 26} and Pten^{loxP/loxP 27} mice were obtained from the NCl mouse depository and intercrossed to generate described genotypes. Primary astrocytes were isolated from the neocortex of 4 day old pups, using standard astrocyte isolation culture conditions²⁸. Conditional deletion of Pten^{loxP/loxP} was accomplished by infecting astrocyte cultures with adenovirus expressing Cre recombinase under the CMV promoter (Gene TransferVector Core, University of Iowa, Iowa City, Iowa). Effective Pten deletion was verified by western blot.

Immunoprecipitations. For mass spectrometry, FLAG-HA-PTEN and V5-src were transiently transfected into 293T cells using Lipofectamine 2000 (Invitrogen Corp. Carlsbad, Calif.). 48 hours post-transfection, cells were lysed in RIPA buffer (50 mM Tris pH 7.5; 150 mM NaCl; 1% NP-40; 0.5% deoxycholate; 0.1% SDS; 1 mM EDTA; 1 mM NaF; phosphatase inhibitor cocktails 1 and 2 (Sigma) and complete protease inhibitor cocktail (Roche). PTEN was immunoprecipitated from 6 mg total protein using M2-agarose (Sigma) for 3 hours at 4° C. Following extensive washing of immunocomplexes in RIPA buffer, PTEN was eluted from the antibody in 5% acetic acid at room temperature. All other PTEN immunoprecipitations were conducted overnight at 4° C. using anti-PTEN goat polyclonal (N-19, Santa Cruz) and protein G-sepharose (GE Healthcare).

Identification of PTEN phosphorylation sites by mass spectrometry. IMAC resin and samples were prepared as described previously²⁹ with the following modifications. The eluted proteins were dried under reduced pressure with 8M Urea/50 mM Tris pH 8.0. The dried sample was resuspended by adding TBS to a final volume of 100 uL. The sample was reduced by DTT, alkylated by iodoacetamide, diluted with TBS to a final concentration of 2M urea, and then digested by 0.5 μg of trypsin at 37° C. overnight. The resulting peptides were desalted using a 50 mg Sep-Pak C18 column (Waters) and dried under vacuum. The dried peptides were resuspended in 20 μl of 1% acetic acid and loaded to a tip-column containing 15 μL of IMAC resin. After loading, the IMAC was washed twice with 15 μL of wash buffer 0.6% acetic acid, once with 20 μl of water, and finally eluted by 100 μL of 6% ammonium hydroxide and dried.

The tandem mass spectra data was collected by an LTQ Orbitrap Discovery using the Xcalibur software version 2.0.7. The raw file was transferred to Sorcerer-SEQUEST system (SageN, San Jose, Calif.). A semi-tryptic peptide monoisotopic database was generated using the human IPI database, version 3.47. The following parameters were applied to the Sorcerer-SEQUEST search: parent mass tolerance of 50 ppm, +79.966331 amu variable modification of STY due to phosphorylation, and maximum of 3 modifications per peptide. The searched data was then analyzed by TPP (Institute for Systems Biology, Seattle, Wash.) and peptides with a minimum probability of 0.8 and Sp rank <5 were considered.

Statistical analysis. For quantitation and analysis of immunohistochemistry data, see above. For analysis of drug sensitivity data, 2-way ANOVA was performed using the KaleidaGraph program (Synergy Software).

### REFERENCES

1. Furnari, F. B. et al. Malignant astrocytic glioma: genetics, biology, and paths to treatment. Genes Dev 21, 2683-710 (2007).
2. Mellinghoff, I. K. et al. Molecular determinants of the response of glioblastomas to EGFR kinase inhibitors. N Engl J Med 353, 2012-24 (2005).
3. Haas-Kogan, D. A. et al. Epidermal growth factor receptor, protein kinase B/Akt, and glioma response to erlotinib. J Natl Cancer Inst 97, 880-7 (2005).
4. Mellinghoff, I. K., Cloughesy, T. F. & Mischel, P. S. PTEN-mediated resistance to epidermal growth factor receptor kinase inhibitors. Clin Cancer Res 13, 378-81 (2007).
5. Speake, G., Holloway, B. & Costello, G. Recent developments related to the EGFR as a target for cancer chemotherapy. Curr Opin Pharmacol 5, 343-9 (2005).
6. Comprehensive genomic characterization defines human glioblastoma genes and core pathways. Nature 455, 1061-8 (2008).
7. Wang, X. & Jiang, X. Post-translational regulation of PTEN. Oncogene 27, 5454-63 (2008).
8. Lu, Y. et al. Src family protein-tyrosine kinases alter the function of PTEN to regulate phosphatidylinositol 3-kinase/AKT cascades. J Biol Chem 278, 40057-66 (2003).
9. Nagata, Y. et al. PTEN activation contributes to tumor inhibition by trastuzumab, and loss of PTEN predicts trastuzumab resistance in patients. Cancer Cell 6, 117-27 (2004).
10. Han, S. Y. et al. Functional evaluation of PTEN missense mutations using in vitro phosphoinositide phosphatase assay. Cancer Res 60, 3147-51 (2000).
11. Koul, D. et al. Motif analysis of the tumor suppressor gene MMAC/PTEN identifies tyrosines critical for tumor suppression and lipid phosphatase activity. Oncogene 21, 2357-64 (2002).
12. Bachoo, R. M. et al. Epidermal growth factor receptor and Ink4a/Arf: convergent mechanisms governing terminal differentiation and transformation along the neural stem cell to astrocyte axis. Cancer Cell 1, 269-77 (2002).
13. de la Iglesia, N. et al. Identification of a PTEN-regulated STAT3 brain tumor suppressor pathway. Genes Dev 22, 449-62 (2008).
14. Dey, N. et al. The protein phosphatase activity of PTEN regulates SRC family kinases and controls glioma migration. Cancer Res 68, 1862-71 (2008).
15. Pandita, A., Aldape, K. D., Zadeh, G., Guha, A. & James, C. D. Contrasting in vivo and in vitro fates of glioblastoma cell subpopulations with amplified EGFR. Genes Chromosomes Cancer 39, 29-36 (2004).
16. Stommel, J. M. et al. Coactivation of receptor tyrosine kinases affects the response of tumor cells to targeted therapies. Science 318, 287-90 (2007).
17. Du, J. et al. Bead-based profiling of tyrosine kinase phosphorylation identifies SRC as a potential target for glioblastoma therapy. Nat Biotechnol 27, 77-83 (2009).
18. Johns, T. G. et al. The efficacy of epidermal growth factor receptor-specific antibodies against glioma xenografts is influenced by receptor levels, activation status, and heterodimerization. Clin Cancer Res 13, 1911-25 (2007).
19. Furnari, F. B., Huang, H. J. & Cavenee, W. K. The phosphoinositol phosphatase activity of PTEN mediates a serum-sensitive G1 growth arrest in glioma cells. Cancer Res 58, 5002-8 (1998).
20. Denning, G., Jean-Joseph, B., Prince, C., Durden, D. L. & Vogt, P. K. A short N-terminal sequence of PTEN controls cytoplasmic localization and is required for suppression of cell growth. Oncogene 26, 3930-40 (2007).
21. Liu, J. L. et al. Nuclear PTEN-mediated growth suppression is independent of Akt down-regulation. Mol Cell Biol 25, 6211-24 (2005).
22. Lo, H. W., Cao, X., Zhu, H. & Ali-Osman, F. Constitutively activated STAT3 frequently coexpresses with epidermal growth factor receptor in high-grade gliomas and targeting STAT3 sensitizes them to Iressa and alkylators. Clin Cancer Res 14, 6042-54 (2008).
23. Nishikawa, R. et al. A mutant epidermal growth factor receptor common in human glioma confers enhanced tumorigenicity. Proc Natl Acad Sci USA 91, 7727-31 (1994).
24. Albuquerque, C. P. et al. A multidimensional chromatography technology for in-depth phosphoproteome analysis. Mol Cell Proteomics 7, 1389-96 (2008).
25. Cloughesy, T. F. et al. Antitumor activity of rapamycin in a Phase I trial for patients with recurrent PTEN-deficient glioblastoma. PLoS Med 5, e8 (2008).
26. Serrano, M. et al. Role of the INK4a locus in tumor suppression and cell mortality. Cell 85, 27-37 (1996).
27. Groszer, M. et al. Negative regulation of neural stem/progenitor cell proliferation by the Pten tumor suppressor gene in vivo. Science 294, 2186-9 (2001).
28. Bachoo, R. M. et al. Epidermal growth factor receptor and Ink4a/Arf: convergent mechanisms governing terminal differentiation and transformation along the neural stem cell to astrocyte axis. Cancer Cell 1, 269-77 (2002).
29. Albuquerque, C. P. et al. A multidimensional chromatography technology for in-depth phosphoproteome analysis. Mol Cell Proteomics 7, 1389-96 (2008).

### Example 2

To further evaluate PTEN in a clinical setting, glioblastoma (GBM) patient samples at initial diagnosis were evaluated for expression of PTEN, PY240PTEN, EGFR and EGFRvIII. The expression of these and overall survival of the patients is noted in Table 2. These patients had received surgical resection, radiotherapy and chemotherapy with BCNU but no targeted cancer or EGFR therapy.

TABLE 2

| n of | overexpression | | total | | overall |
|---|---|---|---|---|---|
| cases | EGFR | EGFRvIII | PTEN | pY240 | Survival (mo) |
| 8 | + | | + | − | 23.88 ± 7.11 |
| 20 | + | | + | + | 10.85 ± 3.25 |
| 11 | − | | + | − | 8.91 ± 7.42 |
| 18 | − | | + | + | 13.39 ± 15.05 |
| 27 | | − | + | + | 15.37 ± 3.84 |
| 17 | | − | + | − | 15.05 ± 16.58 |
| 11 | | + | + | + | 7.18 ± 1.36 |
| 2 | | + | + | − | 16 ± 20.51 |

Mean survival time: includes cases of GBM dead (survival time) and alive (follow up time).

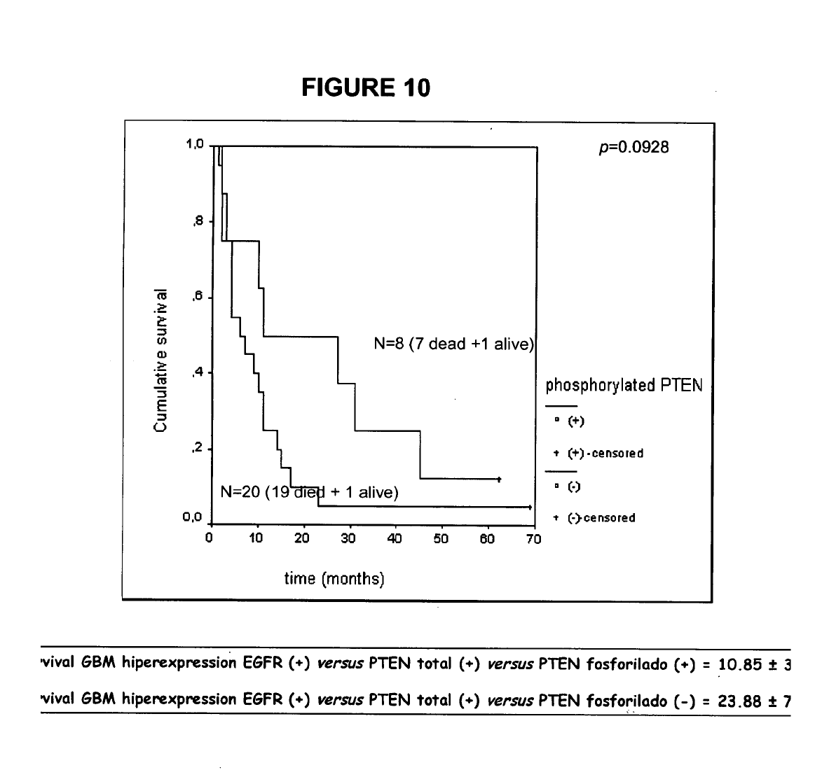

Cumulative survival of glioblastoma patients with EGFR overexpression, PTEN expression and no Y240 PTEN phosphorylation versus those with phosphorylated Y240 PTEN is shown graphically in FIG. 10. Mean survival of patients without PTEN Y240 phosphorylation was 23.88 months, while mean survival of patients with Y240 PTEN phosphorylation was significantly reduced, to 10.85 months. 20 patients with PTEN phosphorylation are already deceased, while only 8 without phosphorylation have deceased. PTEN phosphorylation is correlated with reduced cumulative survival time in glioblastoma patients overexpressing EGFR.

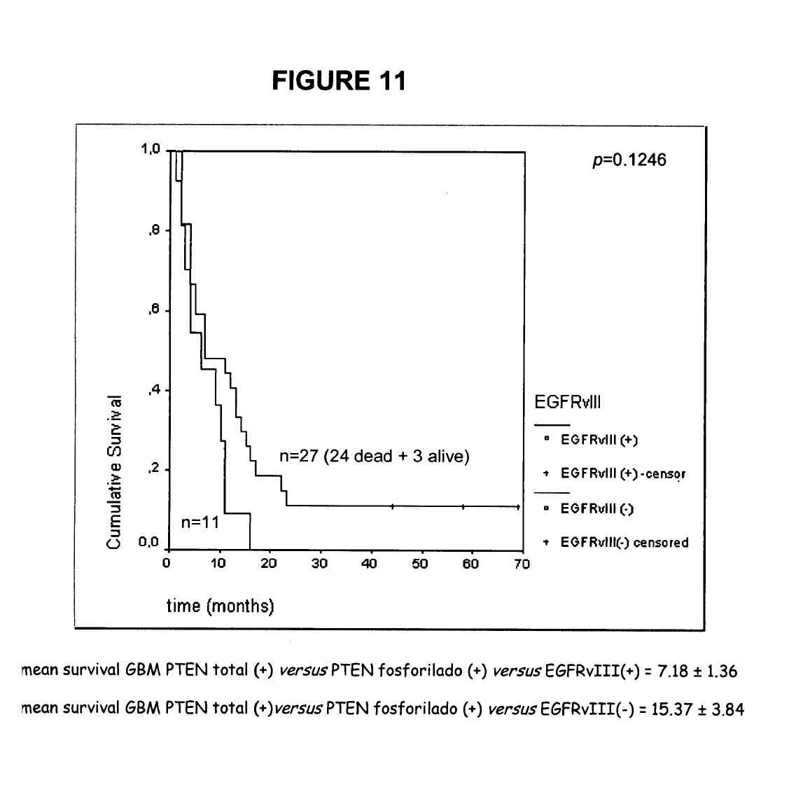

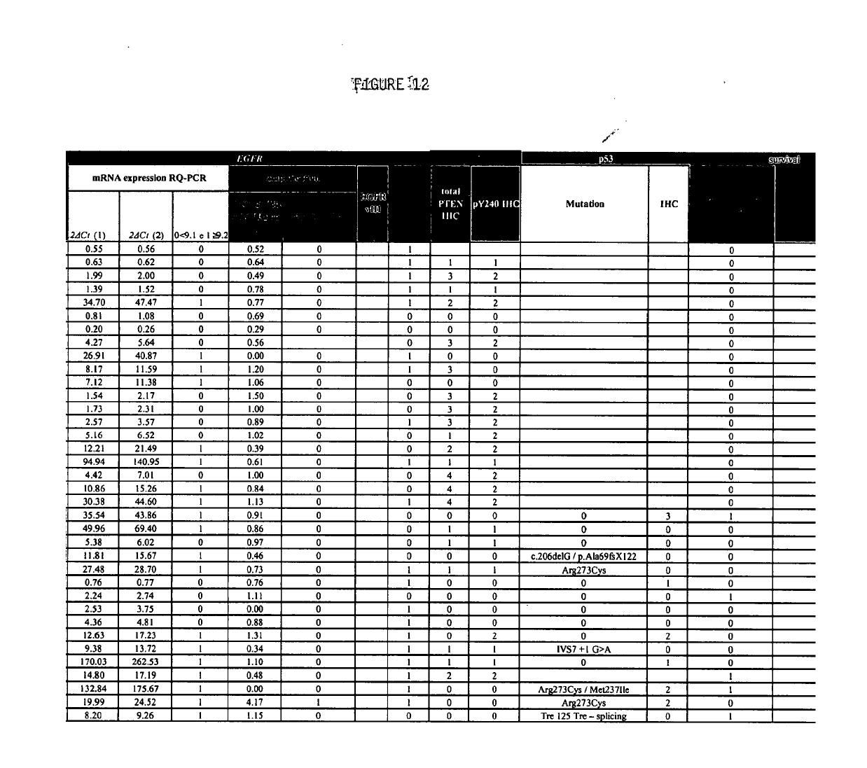

GBM patients with PTEN expression and phosphorylated Y240 PTEN were compared for presence of EGFRvIII mutant and their resultant cumulative survival rates (FIG. 11). Mean survival of patients with PTEN expression, Y240 PTEN phosphorylation, but having wt EGFR and not EGFRvIII was 15.37 months, while mean survival of patients with PTEN expression, Y240 PTEN phosphorylation, and EGFRvIII was 7.18 months. Y240 phosphorylation is correlated with reduced survival in GBM patients. Those patients who also express EGFR mutant vIII demonstrate an even poorer prognosis, with further reduced overall survival.

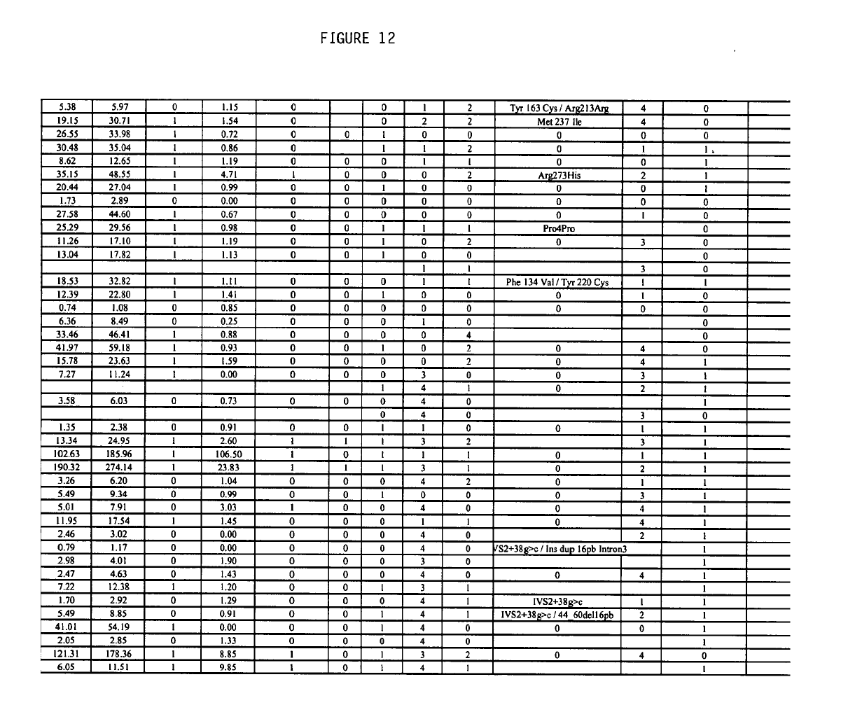

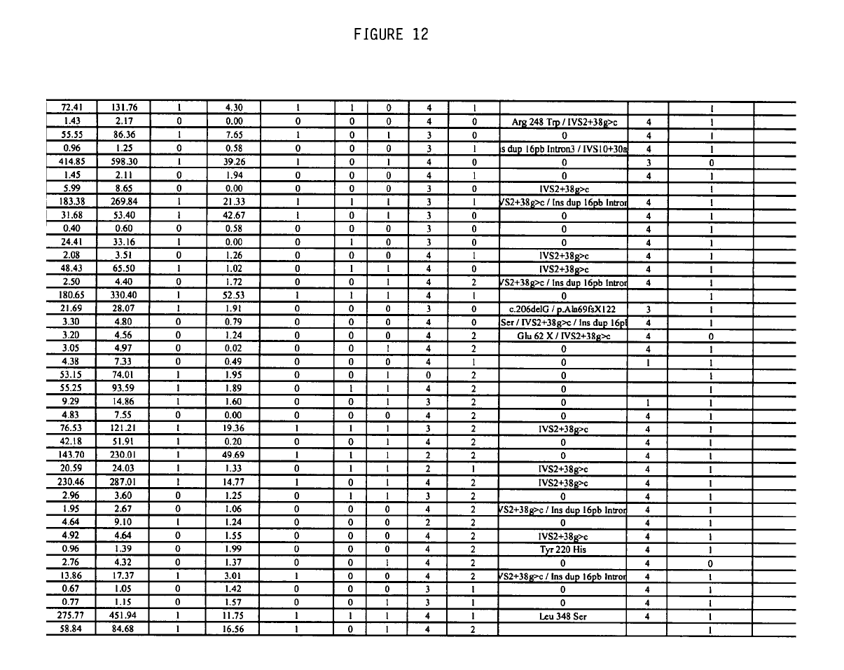

Patients with astrocytomas grades I, II and III and glioblastomas were evaluated, using samples obtained at initial diagnosis, for EGFR, EGFR vIII, total PTEN, phosphorylated Y240 PTEN, and p53 mutations and their current survival status and total survival time. The results are tabulated and presented in FIG. 12.

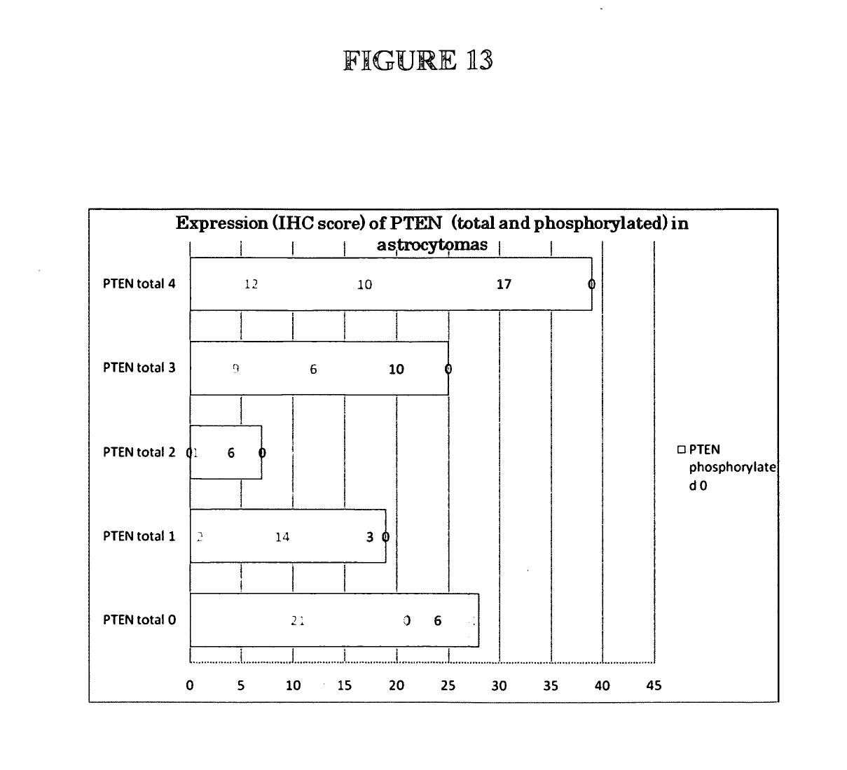

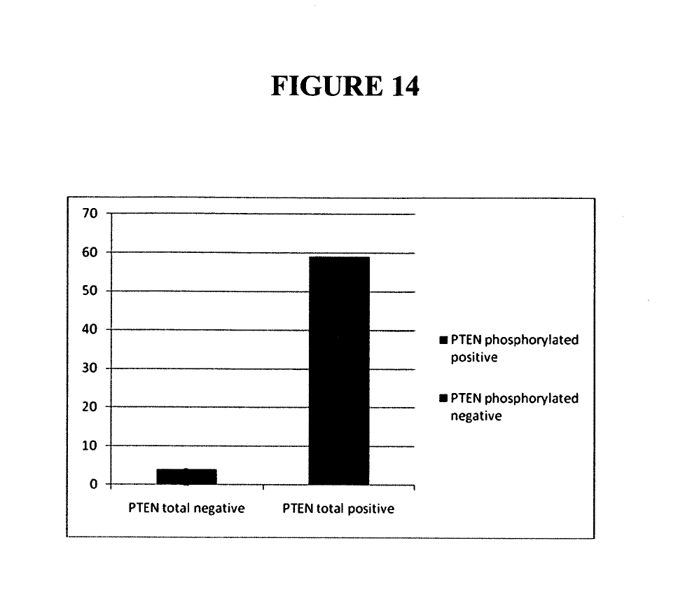

Expression of PTEN total and phosphorylated by immunohistochemistry (IHC) score is tabulated in FIG. 13, with PTEN scores of 0-4 graphed versus number of astrocytoma patients. The number of PTEN positive and negative patients at diagnosis are displayed in FIG. 14. Among PTEN positive, 39 patients were phosphorylated and 20 were negative for phosphorylation. Additional patient data is tabulated below in Tables 3, 4 and 5. Table 3 provides correlations between total PTEM and phosphorylated PTEN versus histopathology, EGFR mRNA expression, EGFR protein expression, EGF deletion status and EGFR amplification status. In Table 5 depicts correlations between total PTEN by IHC score 0-4 versus PTEN phosphorylated IHC score of 0-4. Table 6 provides PTEN total negative or positive by IHC and PTEN phosphorylated negative or positive by IHC versus TP53 mutation and p53 IHC status.

TABLE 3
Correlation between total PTEN and phosphorylated PTEN expression and histopathological characteristics,
EGFR mRNA expression, EGFR protein expression, EGFR deletion and EGFR amplification

| | | PTEN total IHC | | | | | PTEN phosphorylated IHC | | | | |
|---|---|---|---|---|---|---|---|---|---|---|---|
| | N | Negative (%) | | Positive (%) | | | Negative (%) | | Positive (%) | | |
| Pathology | | | | | | | | | | | |
| Non-neoplastic | 3 | 2 | (66.7) | 1 | (33.3) | | 0 | | 3 | (100) | |
| Pilocytic astrocytoma | 16 | 6 | (37.5) | 10 | (62.5) | | 5 | (31.2) | 11 | (68.7) | |
| Low grade | 18 | 16 | (88.9) | 2 | (11.1) | p = 0.000 | 9 | (50) | 9 | (50) | p = 0.431 |
| astrocytoma | | | | | | | | | | | |
| Anaplastic | 18 | 18 | (100) | 0 | | | 9 | (44.4) | 10 | (55.6) | |
| astrocytoma | | | | | | | | | | | |
| Glioblastoma | 63 | 4 | (6.3) | 59 | (93.6) | | 22 | (34.9) | 41 | (65.1) | |
| Total | 118 | | | | | | | | | | |
| EGFR IHC status | | | | | | | | | | | |
| Negative | 58 | 21 | (36.2) | 37 | (63.8) | p = 0.619 | 26 | (44.8) | 32 | (55.2) | p = 0.110 |
| Positive | 59 | 24 | (40.7) | 35 | (59.3) | | 18 | (30.5) | 41 | (69.5) | |
| Total | 117 | | | | | | | | | | |
| EGFR mRNA | | | | | | | | | | | |
| expression | | | | | | | | | | | |
| Low | 51 | 16 | (31.4) | 35 | (68.6) | p = 0.128 | 22 | (43.1) | 29 | (56.9) | p = 0.256 |
| High | 64 | 29 | (45.3) | 35 | (54.7) | | 21 | (32.8) | 43 | (67.2) | |
| Total | 115 | | | | | | | | | | |
| EGFR deletion | | | | | | | | | | | |
| Absent | 64 | 20 | (31.2) | 44 | (68.7) | p = 0.019 | 27 | (42.2) | 37 | (57.8) | p = 0.069 |
| Present | 13 | 0 | | 12 | (100) | | 2 | (15.4) | 11 | (84.6) | |
| Total | 77 | | | | | | | | | | |
| EGFR amplification | | | | | | | | | | | |
| Absent | 95 | 42 | (44.2) | 53 | (55.8) | p = 0.015 | 38 | (40) | 57 | (60) | p = 0.208 |
| Present | 20 | 3 | (15) | 17 | (85) | | 5 | (25) | 15 | (75) | |
| Total | 115 | | | | | | | | | | |

Immunohistochemistry (IHC) staining for PTEN total: negative expression, considered as no staining or less than 24% of structures positively stained; positive expression considered as >25% of structures positively stained.
Phosphorylated PTEN IHC: negative expression = no staining or less than 10% of structures stained; positive expression = >10% os stained structures.
EGFR IHC: negative expression = no staining or less than 24%; positive expression = >25% of structures stained.
EGFR mRNA expression; high expression in comparison to GBM median expression (≧9.1); low expression, according to GBM median (<9.1)
EGFR amplification; present = >2 Ct; absent = ≦2 Ct.

TABLE 4
Correlation between total PTEN and phosphorylated
PTEN expression (IHC score) in astrocytoma

| PTEN phosphorylated | | PTEN total IHC score | | | | |
|---|---|---|---|---|---|---|
| IHC score | N | 0 | 1 | 2 | 3 | 4 |
| 0 | 44 | 21 | 2 | 0 | 9 | 12 |
| 1 | 31 | 0 | 14 | 1 | 6 | 10 |
| 2 | 42 | 6 | 3 | 6 | 10 | 17 |
| 4 | 1 | 1 | 0 | 0 | 0 | 0 |
| Total | 118 | | | | | |

| | PTEN | PTEN | PTEN | PTEN | PTEN |
|---|---|---|---|---|---|
| | total | total 1 | total 2 | total 3 | total 4 |
| PTEN phosphorylated 0 | 21 | 2 | 0 | 9 | 12 |
| PTEN phosphorylated 1 | 0 | 14 | 1 | 6 | 10 |
| PTEN phosphorylated 2 | 6 | 3 | 6 | 10 | 17 |
| PTEN phosphorylated 4 | 1 | 0 | 0 | 0 | 0 |

Immunohistochemistry (IHC) staining for PTEn was divided into score as: 0, <10%; 1, 11-25%; 2, 26-50%; 3, 51-75%; 4, 76-100% of cells staining

TABLE 5
Correlation between total PTEN and phosphorylated PTEN
expression and TP53 mutation, p53 protein expression

| | | PTEN total IHC | | | PTEN phosphorylated IHC | | |
|---|---|---|---|---|---|---|---|
| | N | Negative (%) | Positive (%) | | Negative (%) | Positive (%) | |
| TP53 mutation | | | | | | | |
| Absent | 66 | 24 (36.4) | 42 (63.6) | p = 0.141 | 25 (37.9) | 41 (62.1) | p = 0.938 |
| Present | 18 | 10 (55.6) | 8 (44.4) | | 7 (38.9) | 11 (61.1) | |
| Total | 84 | | | | | | |
| P53 IHC status | | | | | | | |
| Negative | 28 | 22 (78.6) | 6 (21.4) | p = 0.000 | 15 (53.6) | 13 (46.4) | p = 0.052 |
| Positive | 54 | 12 (22.2) | 42 (77.8) | | 17 (31.5) | 37 (68.5) | |
| Total | 82 | | | | | | |

P53 IHC: negative expression = no staining or less than 24%; positive expression = >25% of stained structures

Patients and Methods

Tissue samples. Three non-neoplastic tissues from epilepsy surgeries and 115 tumor samples were collected during surgical procedures by the Neurosurgery Group of the Department of Neurology, Hospital das Clínicas, School of Medicine of São Paulo University, Brazil. Informed consent was obtained from each patient. Fresh surgical samples were immediately snap-frozen in liquid nitrogen upon surgical removal. Before RNA extraction, a 4 μm thick cryosection of each sample was obtained for histological assessment under light microscope after hematoxylin-eosin staining. Necrotic and non-neoplastic areas were removed from the frozen block of tumoral tissue by microdissection prior to RNA extraction.

PTEN and phosphorylated PTEN (PY240) immunohistochemistry staining. For immunohistochemistry (IHC) analysis, paraffin-embedded tissue sections (4 μm) were submitted to deparaffinization, rehydration and endogenous peroxidase activity blocking with 3% hydrogen peroxide in methanol for 10 min. After antigen retrieval with 0.01M Citrate Buffer, pH 6.0, at pressure cooking for 4 min counting from boiling, the sections were incubated with the primary mouse anti-PY240 antibody (described above) diluted 1:50 by overnight incubation at room temperature (18-20° C.). Then, it was applied the NovoLink Maxpolymer detection system (Novocastra, NewCastle upon Tyne, UK) for 30 min at room temperature. Detection of the antibody was performed with 0.6% diaminobenzadine (DAB) (NovoLink). Sections were counterstained with Harris hematoxyilin and mounted. A positive tissue (colon adenocarcinoma) was used to confirm the consistency of the immunostaining and all the samples were stained in a single batch. Analysis was performed by two observers (FDM and SKNM).

For total PTEN reactions, the monoclonal anti-PTEN (Dako, clone 6H2.1) at dilution 1:400 was applied, after antigen retrieval using Tris-Buffer, pH9.0.

Total PTEN and phosphorylated PTEN expressions were scored according to the percentage of structures stained as 0 (no staining), 1 (>0-25%), 2 (26-50%), 3 (51-75%) and 4 (76-100%) of the structures stained. For statistical analysis, scores of 0-1 were defined as no protein expression and scores of 2-4 as positive protein expression for total PTEN, and score 0 as negative staining, and 1-2 as positive for phosphorylated PTEN. For nuclear accumulation it was measured the intensity of immunoreactions, being 1 as weak, 2 as medium, and 3 as strong staining.

Total RNA extraction and cDNA synthesis. Total RNA was extracted from each sample using RNeasy Mini Kit (Qiagen Inc, Hilden, Germany). Evaluation of RNA quantification and purification were carried out by measuring absorbance at 260 and 280 nm. Ratios of 260/280 measures ranging from 1.8 to 2.0 were considered satisfactory for purity standards. Denaturing agarose gel electrophoresis was used to assess the quality of the samples. Synthesis of cDNA was performed by a conventional reverse transcription, using oligo (dT), random primers (Invitrogen, Carlsbad, Calif.) for extension, RNase inhibitor and Super Script III according to the manufacturer's specifications (Invitrogen). The resulting cDNA was then treated with RNase H (GE Healthcare, Piscataway, N.J.) and diluted with TE buffer.

Real time PCR for EGFR expression. The relative expression of EGFR was determined by a quantitative real time PCR (QT-PCR) using the Syber Green I approach. Primers were designed to amplify a 101 bp length amplicon with the following set of primers (5′ to 3′): EGFR F: TGCAGCGATACAGCTCAGACCC (SEQ ID NO: 14), EGFR R: TTTGGGAACGGACTGGTTTATG (SEQ ID NO: 15). Standard curves were established to ensure amplification efficiency and analysis of melting curves demonstrated a single peak for the primers. SYBR Green I amplification mixtures (12 μL) contained 3 μL of cDNA, 6 μL of 2× Power SYBR Green I Master Mix (Applied Biosystems) and 3 μL of mixture forward and reverse primers to a final concentration 200 nM. Reactions were run on ABI Prism 7500 sequence detector (Applied Biosystems). PCR was carried out as follows: 2 min at 50° C., 10 min period of polymerase activation at 95° C., and 40 cycles at 95° C. for 15 sec, and 60° C. for 1 min. Quantitative data were normalized relative to the internal housekeepings controls: hypoxanthine guanine phosphoribosyltransferase gene (HPRT) and beta-glucoronidase gene (GUSB). Primer sequences were as follows (5′ to 3′): HPRT F: TGAGGATTTGGAAAGGGTGT (SEQ ID NO: 16), HPRT R: GAGCACACAGAGGGCTACAA (SEQ ID NO: 17); GUSB F: GAAAATACGTGGTTGGAGAGCTCATT (SEQ ID NO: 18), GUSB R: CCGAGTGAAGATCCCCTTTTTA (SEQ ID NO: 19). The final primers concentration was 200 nM for HPRT and 400 nM for GUSB. The equation 2^{-ΔΔCt} was applied to calculate the relative expression of EGFR in tumor samples versus the mean of non-neoplastic tissues where ΔCt=Ct EGFR gene−mean Ct of housekeeping genes and ΔΔCt=ΔCt tumor−mean ΔCt non-neoplastic tissues (21). The QT-PCR runs for each sample were performed in duplicate and repeated when the Ct values were not similar. The results are presented in log₁₀ scale for better visualization. EGFR expression was scored according to the GBM cases median value=7.5. For statistical analysis, scores ≧7.5 were defined as EGFR overexpression.

Real time PCR for EGFR deletion. The deletion EGFRvIII was detected by QT-PCR to amplify a 352 bp length amplicon (16). Primer sequences were as follows (5′ to 3′): EGFRvIII F: GAGTCGGGCTCTGGAGGAAAAG (SEQ ID NO: 20), EGFRvIII R: CCACAGGCTCGGACGCAC (SEQ ID NO: 21). The final primer concentration used was 800 nM and all reactions were run in duplicate as described for EGFR expression. The deletion analysis was performed by qualitative method and all positive cases were repeated to confirm the result. All primers were synthesized by IDT (Integrated DNA Technologies, Inc, Coraville, Iowa).

Statistical analysis. The non parametric tests were Kruskal-Wallis, Dunn and Spearman correlation once GBM samples have no normal distribution in all tested groups. The survival plot was acquired by Kaplan-Meier test. All the statistical analysis was performed by SSPS, version 15.0 (Chicago, Ill., USA). Differences were considered to be statistically significant at p<0.05.

### Example 3

In the above studies, we established that:

1) PTEN can be phosphorylated on tyrosine 240 upon co-expression with the canonical src-family kinase member, pp 60-src.
2) Tyrosine 240 phosphorylation is correlated with src-family kinase activation in glioblastoma clinical samples.
3) That inhibition of src-family kinases blocks tyrosine 240 phosphorylation in glioblastoma cell lines.

We now set out to specifically identify the src family kinase(s) responsible for Y-240 phosphorylation observed on PTEN. Previous reports have implicated three src family kinases in glioblastoma: src, fyn and lyn (Stettner et al, 2005; Lu et al, 2009). Having failed to detect PTEN phosphorylation in the U87MG cell line, which expresses Fyn and src but not lyn (data not shown), we investigated whether Lyn may be the src family kinase member responsible for the Y240 phosphorylation we observed in clinical samples. Upon co-expression of the Lyn cDNA with PTEN in HEK 293 cells, we observed a robust phosphorylation of PTEN on Y240. While src and lyn are both able to phosphorylate PTEN to a similar extent overall (as assessed by the 4G10 antibody), it appears that lyn exerts a strong preference for the 240 site. Furthermore, PTEN Y240 phosphorylation is enriched in the RIPA-insoluble fraction of these cells, which largely consists of nuclear matrix proteins. Both PTEN and Lyn have been reported to interact with the nuclear matrix, or with matrix-associated proteins (Shen et al, 2007; Radha et al, 1996).

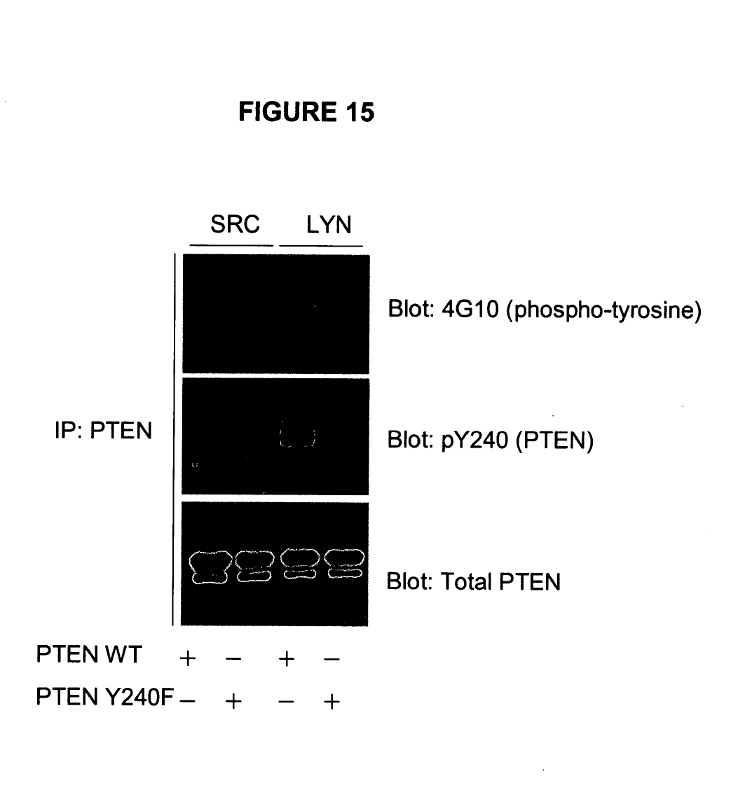

Studies on co-transfection of src or lyn with WT or Y240F PTEN in 293 cells are shown in FIG. 15. This figure demonstrates that although src and lyn both phosphorylate PTEN on tyrosine, the phosphorylation of Y240 is much greater in the presence of lyn than when src is expressed with PTEN. Total PTEN and phosphorylated Y240 PTEN were evaluated on Western blots on cotransfection with src or lyn in combination with wild type (WT) PTEN or PTEN Y240F which cannot be phosphorylated. Lyn is a potent Y240 kinase on PTEN.

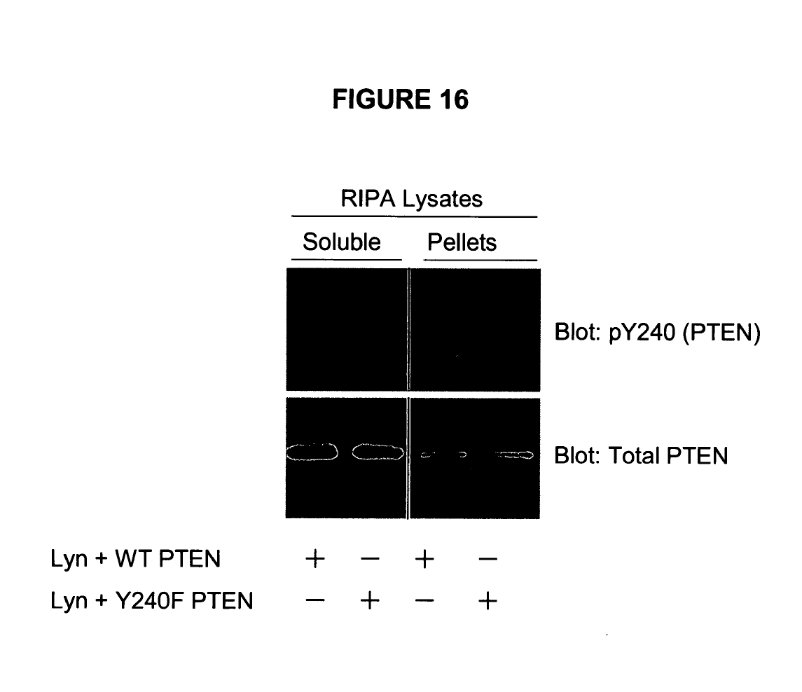

Studies with total lysates from 293 cells expressing Lyn with WT or Y240F PTEN are shown in FIG. 16. Y240 PTEN and total PTEN were evaluated in the soluble versus pellet fraction of 293 cells expressing lyn with WT PTEN or Y240F PTEN. Cells were lysed in RIPA buffer (containing 1% NP40, 0.5% deoxycholate, 0.1% SDS), lysates were passed 10× through an 18 gauge needle then centrifuged at 14,000 rpm. Pellets were washed in RIPA buffer before solubilization in 2×SDS PAGE sample buffer. Y240 phosphorylated PTEN is greatly enriched in the RIPA-insoluble fraction.

Methods:

pCMV-Lyn (Origene cDNA: accession number NM_{—}002350.1) and Flag-HA-PTEN and pCi-Src plasmids (described earlier) were expressed in HEK293 cells by transient transfection in 10 cm dishes using Lipofectamine 2000 (Invitrogen) according to the manufacturers' instructions. At 48 hours post-transfection the cells were washed in ice cold PBS and scraped into 0.5 ml RIPA buffer (50 mM Tris pH 7.5, 150 mM NaCl, 1 mM NaF, 1 mM EDTA, 0.1% SDS, 0.5% Deoxycholate, 1% NP-40 with phosphatase inhibitor cocktails 1 and 2 (Sigma) and complete protease inhibitors (Roche). Lysates were passed 10 times through an 18-gauge needle before centrifugation at 14,000 g for 15 minutes at 4° C. Total protein concentrations were determined by BCA assay (Pierce) and PTEN was immunoprecipitated from 2.5 mg of the cleared supernatants using A2B1 monoclonal antibody (Santa Cruz) and protein-G sepharose (GE Healthcare). Western blots were probed with antibodies to global phosphotyrosine (4G10-HRP, Millipore), PTEN pY240 (described earlier) and total PTEN (A2B1-HRP, Santa Cruz). For detection of PTEN phosphorylation in the RIPA-insoluble fractions, the pellets were solublized by boiling in 40 ul 2×SDS PAGE sample buffer. 10 ul of this material was run per lane on western blots.

For detection of PTEN Y240 phosphorylation in glioma cell lines, GBM39 was grown as described (main methods section) and cells were treated as indicated with 100 nM Dasatinib for one hour prior to treatment with 100 μM Sodium Pervanadate for 15 minutes. Cells were lysed in RIPA buffer as previously and western blots were probed with PTEN pY240 antibody and total PTEN.

The TS543 neurosphere line from Dr. Cameron Brennan (Memorial Sloan Kettering Cancer Center) was propagated in Neurocult Medium (StemCell Techologies) supplemented with bFGF and EGF. Cells were left untreated, or treated with 100 nM Dasatinib for 24 hours prior to lysis in RIPA buffer and detection of PTEN pY240 by western blotting.

### REFERENCES

Lu K V, Zhu S, Cvrljevic A, Huang T T, Sarkaria S, Ahkavan D, Dang J, Dinca E B, Plaisier S B, Oderberg I, Lee Y, Chen Z, Caldwell J S, Xie Y, Loo J A, Seligson D, Chakravari A, Lee F Y, Weinmann R, Cloughesy T F, Nelson S F, Bergers G, Graeber T, Furnari F B, James C D, Cavenee W K, Johns T G, Mischel P S. 2009 ‘Fyn and SRC are effectors of oncogenic epidermal growth factor receptor signaling in glioblastoma patients’ Cancer Res 2009 69(17):6889-98.
Stettner M R, Wang W, Nabors L B, Bharara S, Flynn D C, Grammer J R, Gillespie G Y, Gladson C L. 2005 ‘Lyn kinase activity is the predominant SRC kinase activity in glioblastoma tumor cells’ Cancer Res 65(13):5535-43.
Radha V, Nambirajan S, Swamp G. 1996 ‘Association of Lyn tyrosine kinase with the nuclear matrix and cell cycle-dependent changes in matrix-associated tyrosine kinase activity’ Eur J Biochem 236(2):352-9
Shen W H, Balajee A S, Wang J, Wu H, Eng C, Pandolfi P P, Yin Y. 2007 ‘Essential role for nuclear PTEN in maintaining chromosome integrity’ Cell 128(1):157-70
   This invention may be embodied in other forms or carried out in other ways without departing from the spirit or essential characteristics thereof. The present disclosure is therefore to be considered as in all aspects illustrated and not restrictive, the scope of the invention being indicated by the appended Claims, and all changes which come within the meaning and range of equivalency are intended to be embraced therein.
   Various references are cited throughout this Specification, each of which is incorporated herein by reference in its entirety.

What is claimed is:

1. A method for determining sensitivity or resistance to a therapeutic agent in a cancer patient comprising detecting tyrosine phosphorylation of PTEN in a tumor biopsy or cancer cell sample.

2. The method of claim 1 wherein Y240 PTEN phosphorylation is determined and the presence of Y240 phosphorylation indicates reduced sensitivity or resistance to a therapeutic agent.

3. The method of claim 1 wherein phosphorylation of PTEN is determined by binding or recognition of a phosphospecific antibody.

4. The method of claim 3 wherein phosphorylation of PTEN is determined at Y240 of PTEN by binding or recognition of a phosphospecific antibody which specifically recognizes tyrosine 240 (Y240) specific phosphorylation of PTEN.

5. The method of claim 2 wherein the therapeutic agent is an EGFR kinase inhibitor and the presence of Y240 phosphorylation indicates reduced sensitivity or resistance to said EGFR inhibitor.

6. A method for determining sensitivity or resistance to an EGFR inhibitor in a cancer patient comprising detecting tyrosine phosphorylation of PTEN in a tumor biopsy or cancer cell sample.

7. The method of claim 6 wherein phosphorylation of PTEN is determined by binding or recognition of a phosphospecific antibody.

8. The method of claim 7 wherein phosphorylation of PTEN is determined at Y240 of PTEN by binding or recognition of a phosphospecific antibody which specifically recognizes tyrosine 240 (Y240) specific phosphorylation of PTEN.

9. The method of claim 1 or 6 wherein the cancer is selected from brain, melanoma, breast, prostate, renal, endometrial, lung, stomach, colon, and kidney.

10. The method of claim 9 wherein the cancer is brain cancer and is astrocytoma.

11. The method of claim 10 wherein the cancer is glioblastoma.

12. A method of determining the prognosis of cancer in a mammal comprising detecting tyrosine 240 phosphorylation of PTEN in a tumor biopsy or cancer cell sample from said mammal, wherein Y240 PTEN phosphorylation is indicative of a poor prognosis or reduced survival time on cancer diagnosis in said mammal.

13. The method of claim 12 wherein the cancer is an ErbB family mediated cancer.

14. The method of claim 12 wherein the cancer is an EGFR-mediated cancer.

15. The method of claim 12 further comprising detecting EGFR expression and/or determining the presence of EGFR vIII, wherein Y240 PTEN phosphorylation, combined with increased EGFR expression and the presence of EGFR vIII is indicative of a poor prognosis or reduced survival time on cancer diagnosis in said mammal.

16. The method of claim 12 wherein the cancer is selected from brain, melanoma, breast, prostate, renal, endometrial, lung, stomach, colon, and kidney.

17. The method of claim 16 the cancer is brain cancer and is astrocytoma.

18. The method of claim 17 wherein the cancer is glioblastoma.

19. A method of monitoring and treating cancer in a mammal comprising:
(a) detecting tyrosine phosphorylation of PTEN in a tumor biopsy or cancer cell sample of said mammal and determining whether Y240 of PTEN is phosphorylated;
(b) administering a src-family kinase inhibitor alone or in combination with one or more other cancer therapeutic agent to any said mammal wherein Y240 of PTEN is phosphorylated; and
(c) optionally monitoring Y240 PTEN phosphorylation in said mammal after administration of the inhibitor.

20. The method of claim 19 wherein phosphorylation at Y240 of PTEN is determined by binding or recognition of a phosphospecific antibody which specifically recognizes tyrosine 240 (Y240) specific phosphorylation of PTEN.

21. A kit for therapeutic monitoring or prognosis of cancer in an EGFR-mediated cancer, said kit comprising a phosphospecific anti-PTEN antibody or fragment thereof which recognizes tyrosine 240 (Y240) specific phosphorylation of PTEN and does not react with PTEN which lacks phosphorylation at Y240, optionally with reagents and/or instructions for use.

22. A phosphospecific antibody molecule or fragment thereof which recognizes tyrosine 240 (Y240) specific phosphorylation of PTEN and does not react with PTEN which lacks phosphorylation at Y240.

23. The antibody of claim 22 which is a polyclonal antibody.

24. The antibody of claim 22 which is a monoclonal antibody.

25. A method for inducing sensitivity of a cancer to an ErbB inhibitor where said cancer has acquired resistance to the ErbB inhibitor by Y240 phosphorylation of PTEN, comprising administering a combination of a modulator that blocks phosphorylation of PTEN or blocks the downstream signal which results from PTEN phosphorylation.

26. The method of claim 25 for inducing sensitivity of a cancer to an EGFR inhibitor where said cancer has acquired resistance to the EGFR inhibitor by Y240 phosphorylation of PTEN, comprising administering a combination of a modulator that blocks phosphorylation of PTEN or blocks the downstream signal which results from PTEN phosphorylation.

27. The method of claim 25 wherein the modulator that blocks phosphorylation of PTEN is a Src kinase inhibitor and/or a Lyn kinase inhibitor.

28. A method of treating an ErbB family phospho Y240 PTEN positive tumor in a mammal comprising administering a src family targeted tyrosine kinase inhibitor, followed by or in combination with treatment with an ErbB family targeted tyrosine kinase inhibitor.

29. The method of claim 28 for treating an EGFR overexpressing or mutant phospho Y240 PTEN positive tumor in a mammal comprising administering a src family targeted tyrosine kinase inhibitor, followed by or in combination with treatment with an EGFR targeted tyrosine kinase inhibitor.

30. The method of claim 25 or 28 wherein the src family targeted tyrosine kinase inhibitor is Dasatinib or INNO-406.


SEQUENCE LISTING (21 sequences)

SEQ ID NO: 1 - protein, 17 aa, Artificial Sequence
  reverse primer
FMYFEFPQPLPVCGDIK

SEQ ID NO: 2 - protein, 16 aa, Artificial Sequence
  phosphorylated peptide
RLEGVYRNNIDDVVRF

SEQ ID NO: 3 - protein, 10 aa, Artificial Sequence
  phosphorylated peptide
KIYNLCAERH

SEQ ID NO: 4 - protein, 10 aa, Artificial Sequence
  phosphorylated peptide
KIYNLCAERH

SEQ ID NO: 5 - protein, 14 aa, Artificial Sequence
  phosphorylated peptide
KNHYKIYNLCAERH

SEQ ID NO: 6 - protein, 14 aa, Artificial Sequence
  phosphorylated peptide
KAQEALDFYGEVRT

SEQ ID NO: 7 - protein, 14 aa, Artificial Sequence
  phosphorylated peptide
KAQEALDFYGEVRT

SEQ ID NO: 8 - protein, 12 aa, Artificial Sequence
  phosphorylated peptide
RYVYYYSYLLKN

SEQ ID NO: 9 - protein, 19 aa, Artificial Sequence
  phosphorylated peptide
KFMYFEFPQPLPVCGDIKV

SEQ ID NO: 10 - protein, 16 aa, Artificial Sequence
  phosphorylated peptide
RADNDKEYLVLTLTKN

SEQ ID NO: 11 - protein, 31 aa, Artificial Sequence
  phosphorylated peptide
KTVEEPSNPEASSSTSVTPDVSDNEPDHYRY

SEQ ID NO: 12 - protein, 26 aa, Artificial Sequence
  phosphorylated peptide
RYSDTTDSDPENEPFDEDQHTQITKV

SEQ ID NO: 13 - protein, 19 aa, Artificial Sequence
  phosphorylated peptide
TRREDKFMYFEFPQPLPVC

SEQ ID NO: 14 - DNA, 22 bp, Artificial Sequence
  forward primer
tgcagcgatacagctcagaccc

SEQ ID NO: 15 - DNA, 22 bp, Artificial Sequence
  reverse primer
tttgggaacggactggtttatg

SEQ ID NO: 16 - DNA, 20 bp, Artificial Sequence
  forward primer
tgaggatttggaaagggtgt

SEQ ID NO: 17 - DNA, 20 bp, Artificial Sequence
  reverse primer
gagcacacagagggctacaa

SEQ ID NO: 18 - DNA, 26 bp, Artificial Sequence
  forward primer
gaaaatacgtggttggagagctcatt

SEQ ID NO: 19 - DNA, 22 bp, Artificial Sequence
  reverse primer
ccgagtgaagatccccttttta

SEQ ID NO: 20 - DNA, 22 bp, Artificial Sequence
  forward primer
gagtcgggctctggaggaaaag

SEQ ID NO: 21 - DNA, 18 bp, Artificial Sequence
  reverse primer
ccacaggctcggacgcac